(19) United States
(12) Patent Application Publication
(10) Pub. No.: US 2025/0246262 A1
(43) Pub. Date: Jul. 31, 2025

(54) Biomolecule Fitness Inference Using Machine Learning for Drug Discovery with Directed Evolution

(71) Applicant: Genentech, Inc., South San Francisco, CA (US)

(72) Inventors: Tommaso Biancalani, San Francisco, CA (US); Christian Nathaniel Cunningham, Oakland, CA (US); Nathaniel Lee Diamant, Menlo Park, CA (US); Gabriele Scalia, San Mateo, CA (US); Max Walt Shen, San Francisco, CA (US)

(21) Appl. No.: 19/183,041

(22) Filed: Apr. 18, 2025

Related U.S. Application Data
(63) Continuation of application No. PCT/US23/77319, filed on Oct. 19, 2023.
(60) Provisional application No. 63/417,961, filed on Oct. 20, 2022.

(51) Int. Cl. G16B 20/30; G16B 30/00; G16B 40/20

(52) U.S. Cl. CPC G16B 20/30; G16B 30/00; G16B 40/20

(57) ABSTRACT

In one embodiment, a method includes accessing a biomolecule representation of a first biomolecule and processing the biomolecule representation by a machine-learning model trained using sequencing time-series data. The sequencing time-series data was obtained from directed evolution of a population of biomolecules over multiple enrichment rounds where the population of biomolecules in each enrichment round was a unique set of biomolecules with respect to each other enrichment round. The sequencing time-series data for each enrichment round comprises a biomolecule frequency of each biomolecule of the population of biomolecules in the respective enrichment round. The training comprises learning inferred fitness scores of the population of biomolecules for each enrichment round by predicting biomolecule frequencies of the population of biomolecules in the respective enrichment round given biomolecule frequencies of the population of biomolecules in prior enrichment rounds. The method further includes outputting an inferred fitness score for the first biomolecule.

### PRIORITY

This application is a continuation under 35 U.S.C. §365(c) of International Patent Application No. PCT/US2023/077319, filed 19 Oct. 2023, which claims the benefit under 35 U.S.C. §119(e) of U.S. Provisional Patent Application No. 63/417,961, filed 20 Oct. 2022, each of which is incorporated herein by reference.

### TECHNICAL FIELD

This disclosure generally relates to systems and methods for inferring biomolecule fitness, and more specifically to machine-learning techniques for biomolecule selection.

### BACKGROUND

Directed evolution, with iterated mutation and human-designed selection, is a powerful approach for drug discovery, such as large molecule drug discovery. Mutation is an important part of directed evolution. Directed evolution approaches for drug discovery use genetic strategies (e.g., DNA-encoded, RNA-encoded, or phage-based) to create very large but specific libraries of molecules whose amplification is driven by the target of interest. In other words, directed evolution approaches can discover drug-like biomolecules, such as macrocycles, with novel activities of interest.

Current multiplexed target-binding candidate screening analysis systems have difficulty with the simultaneous selection of many nucleotide-containing peptide libraries for binding to a desired target due to problems such as sample-to-sample variations and data complexity. There is, therefore, a need for improved multiplexed target-binding candidate screening analysis systems and methods to help simultaneous selection of candidate binders against a desired binding target, e.g., a protein.

### SUMMARY OF PARTICULAR EMBODIMENTS

Herein is provided a system and methods for biomolecule fitness inference. Challenges exist in how to select diverse biomolecules with improved binding capabilities from directed evolution experiments. One solution is to utilize machine-learning techniques to infer biomolecule fitness based on sequencing time-series data obtained from directed evolution experiments and then select biomolecules based on the inferred fitness.

In particular embodiments, a molecule discovery system described herein utilizes deep-learning techniques for biomolecule fitness selection. As an example and not by way of limitation, the molecule discovery system may include a deep neural network that estimates molecule fitness, representing at least the biological activity of the molecules, and use the fitness to rank biomolecules (e.g., macrocyclic peptides, small molecules, other types of peptide therapeutics such as bicyclic peptides, or any molecule that can be associated with or tagged to a DNA encoded library or other similar technology) for selection. The molecule discovery system may establish a fitness inference problem given on-target and off-target time series DNA sequencing data. The molecule discovery system may utilize maximum likelihood solutions for the nonlinear dynamical system induced by fitness-based competition. The disclosed approach may learn from multiple time series rounds in a principled manner, in contrast to prior work focused on two-round enrichment prediction. By ranking molecules based on fitness, the molecule discovery system can identify low-frequency, high-fitness molecules that may otherwise be missed by conventional systems. The molecule discovery system may additionally predict on-target and off-target binding fitness accurately, discover novel and diverse genotypes of the biomolecules, and improve the quantity and diversity of identified molecules. The experiments show that inferring fitness while jointly learning a sequence-to-fitness deep-learning model (e.g., a transformer) improves performance over a baseline model without deep learning and a two-round enrichment baseline.

In particular embodiments, the molecule discovery system may access a biomolecule representation of a first biomolecule. The molecule discovery system may then process, by a machine-learning model, the biomolecule representation of the first biomolecule. The machine-learning model was trained using sequencing time-series data associated with biomolecule frequencies of particular biomolecules. The sequencing time-series data was obtained from a directed evolution of a population of biomolecules over a plurality of enrichment rounds. The population of biomolecules in each enrichment round may be a unique set of biomolecules with respect to each other enrichment round. As used herein, “unique” indicates that the population of biomolecules may have mutated in each enrichment round, thereby resulting in the population of biomolecules in each enrichment round having at least some unique biomolecules with respect to the population of biomolecules in each other enrichment round. The sequencing time-series data for each enrichment round may comprise a biomolecule frequency of each biomolecule of the population of biomolecules in the respective enrichment round. The training may comprise learning inferred fitness scores of the population of biomolecules for each enrichment round by predicting biomolecule frequencies of the population of biomolecules in the respective enrichment round given biomolecule frequencies of the population of biomolecules in one or more prior enrichment rounds. The molecule discovery system may further output, by the machine-learning model based on the processing of the biomolecule representation of the first biomolecule, an inferred fitness score for the first biomolecule.

Certain technical challenges exist for biomolecule fitness selection. One technical challenge may include effectively establishing a fitness inference task for biomolecules undergoing mutations in enrichment rounds. The solution presented by the embodiments disclosed herein to address this challenge may be developing a model of evolutionary dynamics which optimizes the inferred fitness given the biomolecule frequency as such model accounts for the genetic drift and mutational process of the biomolecules in the enrichment rounds. Another technical challenge may include disentangling the sequencing time-series data by both on-target binding strength and off-target binding. The solution presented by the embodiments disclosed herein to address this challenge may be accounting for off-target fitness, inferring total fitness from standard directed evolution data, and then inferring on-target fitness, as this approach may enable reasoning about the relative contributions of on-target and off-target fitness. Another technical challenge may include the measured enrichment being less reliable for lower counts due to high assay noise. The solution presented by the embodiments disclosed herein to address this challenge may be using a Dirichlet-multinomial loss to optimize the fitness inference task as the Dirichlet-multinomial loss may account for the increased difficulty of predicting biomolecule frequency at a current round from a prior round when the total read counts are lower.

Certain embodiments disclosed herein may provide one or more technical advantages. A technical advantage of the embodiments may include identifying the fitness of biomolecules not in the original enrichment rounds as the molecule discover system utilizes a deep learning model that can infer fitness for any unseen biomolecules. Another technical advantage of the embodiments may include more effective selection of discovered hits as the molecule discover system infers fitness of biomolecules that indicates biological activity and then determines discovered hits based on such biological activity. Another technical advantage of the embodiments may include the ability to filter out low specificity binders from the results of the selection as the molecule discover system may determine both on-target and off-target bindings. Another technical advantage of the embodiments may include the ability to discover novel and diverse genotypes as the molecule discovery system may identify genotypes with high fitness but low frequency (and vice versa), which may be missed through standard approaches for biomolecule discovery. Certain embodiments disclosed herein may provide none, some, or all of the above technical advantages. One or more other technical advantages may be readily apparent to one skilled in the art in view of the figures, descriptions, and claims of the present disclosure.

In particular embodiments, the techniques described herein relate to a method including, by one or more computing systems: accessing a biomolecule representation of a first biomolecule; processing, by a machine-learning model, the biomolecule representation of the first biomolecule, wherein the machine-learning model was trained using sequencing time-series data associated with biomolecule frequencies of particular biomolecules, wherein the sequencing time-series data was obtained from directed evolution of a population of biomolecules over a plurality of enrichment rounds, wherein the population of biomolecules in each enrichment round was a unique set of biomolecules with respect to each other enrichment round, wherein the sequencing time-series data for each enrichment round includes a biomolecule frequency of each biomolecule of the population of biomolecules in the respective enrichment round, and wherein the training includes learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and outputting, by the machine-learning model based on the processing of the biomolecule representation of the first biomolecule, an inferred fitness score for the first biomolecule.

In particular embodiments, the techniques described herein relate to a method, further including: determining, based on the inferred fitness score for the first biomolecule, whether a biological activity associated with the first biomolecule meets a predetermined criteria for selection.

In particular embodiments, the techniques described herein relate to a method, wherein the plurality of enrichment rounds includes at least three enrichment rounds, and wherein at least one of the enrichment rounds was a control round where the population of biomolecules is analyzed without a presence of a target protein.

In particular embodiments, the techniques described herein relate to a method, wherein the inferred fitness score for the first biomolecule indicates a biological activity of the first biomolecule with respect to a target protein.

In particular embodiments, the techniques described herein relate to a method, wherein learning the inferred fitness scores in the training of the machine-learning model includes optimizing a Dirichlet-multinomial loss function, and wherein the Dirichlet-multinomial loss function utilizes an over-dispersed multinomial distribution to account for an increased difficulty associated with predicting biomolecule frequencies of the population of biomolecules in each enrichment round given biomolecule frequencies of the population of biomolecules in a prior enrichment round.

In particular embodiments, the techniques described herein relate to a method, wherein the training of the machine-learning model further includes calculating a Dirichlet loss negative log-likelihood between the predicted biomolecule frequencies and actual biomolecule frequencies as a negative log-likelihood.

In particular embodiments, the techniques described herein relate to a method, wherein the inferred fitness score for the first biomolecule includes an on-target fitness score associated the first biomolecule binding to a target protein.

In particular embodiments, the techniques described herein relate to a method, wherein the inferred fitness score for the first biomolecule includes an off-target fitness score associated the first biomolecule binding to a test instrument instead of a target protein.

In particular embodiments, the techniques described herein relate to a method, wherein the inferred fitness score for the first biomolecule includes an on-target fitness score associated the first biomolecule binding to a target protein and an off-target fitness score associated the first biomolecule binding to a test instrument instead of the target protein, wherein the method further includes: determining a binding specificity of the first biomolecule based on a ratio of the on-target fitness score to the off-target fitness score.

In particular embodiments, the techniques described herein relate to a method, wherein the machine-learning model includes one or more neural networks.

In particular embodiments, the techniques described herein relate to a method, wherein the one or more neural networks include: a first neural network trained for predicting on-target fitness scores associated with biomolecules, and a second neural network trained for predicting off-target fitness scores associated with biomolecules.

In particular embodiments, the techniques described herein relate to a method, further including: generating the biomolecule representation of the first biomolecule, wherein the first biomolecule is a polypeptide corresponding to a first genotype, and wherein the generating includes: determining a plurality of amino acids of the first biomolecule; applying, for each amino acid of the plurality of amino acids, a function to determine a feature representation for the respective amino acid; and generating a genotype representation corresponding to the first genotype based on the plurality of feature representations associated with the plurality of amino acids.

In particular embodiments, the techniques described herein relate to a method, wherein the first biomolecule is within the population of biomolecules in the plurality of enrichment rounds.

In particular embodiments, the techniques described herein relate to a method, wherein the first biomolecule is not within the population of biomolecules in the plurality of enrichment rounds.

In particular embodiments, the techniques described herein relate to a method, wherein the sequencing time-series data include DNA sequencing time-series data, and wherein the biomolecule frequencies of particular biomolecules indicate genotype frequencies.

In particular embodiments, the techniques described herein relate to a method, further including: processing a plurality of biomolecule representations associated with a plurality of respective second biomolecules by the machine-learning model to determine a plurality of inferred fitness scores for the plurality of second biomolecules, respectively; and selecting, based on the inferred fitness scores for the plurality of second biomolecules, one or more second biomolecules meeting a predetermined criteria for selection, wherein one or more of the selected second biomolecules are each associated with a low relative biomolecule frequency in a last round of the plurality of enrichment rounds.

In particular embodiments, the techniques described herein relate to a method, further including: generating a genotype space based on the biomolecule frequencies and the inferred fitness scores for the plurality of second biomolecules; and selecting the one or more second biomolecules by identifying the one or more second biomolecules from one or more regions in the genotype space, wherein each of the one or more regions is associated with a particular biomolecule frequency range and a particular biomolecule fitness range.

In particular embodiments, the techniques described herein relate to a method, wherein the training further includes pretraining an off-target model, including identifying one or more off-target enrichment rounds from the plurality of enrichment rounds; and pretraining the off-target model based on sequencing time-series data for the one or more off-target enrichment rounds.

In particular embodiments, the techniques described herein relate to a method, wherein the training further includes accessing sequencing time-series data from one or more on-target enrichment rounds from the plurality of enrichment rounds; and generating an on-target model based on the accessed sequencing time-series data from the one or more on-target enrichment rounds and the off-target model.

In particular embodiments, the techniques described herein relate to a method, wherein the first biomolecule is a macrocycle.

In particular embodiments, the techniques described herein relate to a method, wherein the population of biomolecules are amplified by polymerase chain reaction (PCR) in each of the plurality of enrichment rounds.

In particular embodiments, the techniques described herein relate to a method, further including: processing a plurality of biomolecule representations associated with a plurality of respective second biomolecules by the machine-learning model to determine a plurality of inferred fitness scores for the plurality of second biomolecules, respectively; and selecting, based on the inferred fitness scores for the plurality of second biomolecules, one or more diverse biomolecules from the plurality of second biomolecules, wherein the one or more diverse biomolecules meet a predetermined criteria for selection, and wherein one or more of the diverse biomolecules are each associated with a low relative biomolecule frequency in a last round of the plurality of enrichment rounds.

In particular embodiments, the techniques described herein relate to one or more computer-readable non-transitory storage media embodying software that is operable when executed to: access a biomolecule representation of a first biomolecule; process, by a machine-learning model, the biomolecule representation of the first biomolecule, wherein the machine-learning model was trained using sequencing time-series data associated with biomolecule frequencies of particular biomolecules, wherein the sequencing time-series data was obtained from a directed evolution of a population of biomolecules over a plurality of enrichment rounds, wherein the population of biomolecules in each enrichment round was a unique set of biomolecules with respect to each other enrichment round, wherein the sequencing time-series data for each enrichment round includes a biomolecule frequency of each biomolecule of the population of biomolecules in the respective enrichment round, and wherein the training includes learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and output, by the machine-learning model based on the processing of the biomolecule representation of the first biomolecule, an inferred fitness score for the first biomolecule.

In particular embodiments, the techniques described herein relate to a system including: one or more processors; and a non-transitory memory coupled to the processors including instructions executable by the processors, the processors operable when executing the instructions to: access a biomolecule representation of a first biomolecule; process, by a machine-learning model, the biomolecule representation of the first biomolecule, wherein the machine-learning model was trained using sequencing time-series data associated with biomolecule frequencies of particular biomolecules, wherein the sequencing time-series data was obtained from a directed evolution of a population of biomolecules over a plurality of enrichment rounds, wherein the population of biomolecules in each enrichment round was a unique set of biomolecules with respect to each other enrichment round, wherein the sequencing time-series data for each enrichment round includes a biomolecule frequency of each biomolecule of the population of biomolecules in the respective enrichment round, and wherein the training includes learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and output, by the machine-learning model based on the processing of the biomolecule representation of the first biomolecule, an inferred fitness score for the first biomolecule.

In particular embodiments, the techniques described herein relate to a method including, by one or more computing systems: accessing a plurality of biomolecule representations of a plurality of respective biomolecules; processing, by a machine-learning model, the plurality of biomolecule representations of the plurality of respective biomolecules, wherein the machine-learning model was trained using sequencing time-series data associated with biomolecule frequencies of particular biomolecules, wherein the sequencing time-series data was obtained from directed evolution of a population of biomolecules over a plurality of enrichment rounds, wherein the population of biomolecules in each enrichment round was a unique set of biomolecules with respect to each other enrichment round, wherein the sequencing time-series data for each enrichment round includes a biomolecule frequency of each biomolecule of the population of biomolecules in the respective enrichment round, and wherein the training includes learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; outputting, by the machine-learning model based on the processing of the plurality of biomolecule representation of the plurality of biomolecules, a plurality of inferred fitness scores for the plurality of biomolecules, respectively; and selecting, based on the inferred fitness scores for the plurality of biomolecules, one or more biomolecules meeting a predetermined criteria for selection, wherein one or more of the selected biomolecules are each associated with a low relative biomolecule frequency in a last round of the plurality of enrichment rounds.

In particular embodiments, the techniques described herein relate to a method, further comprising: generating a genotype space based on the biomolecule frequencies and the inferred fitness scores for the plurality of biomolecules, wherein selecting the one or more biomolecules meeting the predetermined criteria for selection comprises identifying the one or more biomolecules from one or more regions in the genotype space, wherein each of the one or more regions is associated with a particular biomolecule frequency range and a particular biomolecule fitness range.

The embodiments disclosed herein are only examples, and the scope of this disclosure is not limited to them. Particular embodiments may include all, some, or none of the components, elements, features, functions, operations, or steps of the embodiments disclosed herein. Embodiments according to the invention are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

FIG. 1 depicts a system diagram illustrating an example of a molecule discovery system, in accordance with some example embodiments.

FIG. 2A illustrates an example deep-learning fitness model with off-target oracle.

FIG. 2B illustrates another example deep-learning fitness model.

FIG. 3 illustrates an example distribution of the enrichment for each pair of rounds.

FIG. 4 illustrates example on-target fitness versus off-target fitness regarding predictions on 100,000 holdout genotypes.

FIG. 5A illustrates an example genotype space generated based on last-round frequency and last-round frequency winners.

FIG. 5B illustrates an example genotype space generated based on fitness and fitness winners.

FIG. 5C illustrates an example last-round frequency versus fitness.

FIG. 6 illustrates an example method for biomolecule fitness inference.

FIG. 7 illustrates an example computer system.

### DESCRIPTION OF EXAMPLE EMBODIMENTS

### Introduction

Macrocycles are a promising class of drug candidates that are an intermediate between small and large molecules. One protocol uses DNA encoded libraries (DELs) to discover macrocycles with very large library sizes up to 10 trillion. This protocol may enable coupling any DNA codon with any amino acid (aa), natural or non-natural, by codon tables that are constructed by scientists. This known 1:1 mapping allows next-generation sequencing (NGS) readout of the macrocycle peptide's amino acids. To discover hits, libraries undergo multiple rounds of selection involving iterative steps of incubation with the target protein, washing off non-binders, amplification, and re-translating DNA sequences to macrocycle peptides. The amplification may be with intentionally high error rate to introduce mutations during intermediate selection steps. This protocol can be intensive, and its complexity may complicate a probabilistic model of observations. The molecule that undergoes selection in the aforementioned protocol may be a peptide, a p-linker, and DNA.

In addition, there may be a particular interest in identifying shorter macrocycle hits (<9 aa), since they are more likely to be cell permeable, but they are also much weaker (specifically, weaker binding affinity) in binding with the target protein than long macrocycles (10-14 aa). Short macrocycles tend to be less diverse. While traditional methods may have reasonable potency, drug-likeness and cell permeability may be optimized afterwards, which can be challenging. Aa a result, it may be advantageous to first find hits that are cell-permeable and drug-like, and later optimize potency. With traditional methods, selections can be run only with short macrocycles, but the selection may be too harsh as no hits have any meaningful enrichment. Noise instead may dominate, and the signal-to-noise ratio can be poor. One particular kind of “noise” may be that the peptide-DNA linker binds non-specifically to the target protein. For example, the linker enrichment may be about 0.1%, which may be acceptable for large macrocycles with peak enrichment >1% (10:1 signal-to-noise ratio). However, short macrocycles may have peak enrichment of about 0.1% (1:1 SNR), which means the amplification ability of each short macrocycle may be “obscured” by the linker—weak binders would still amplify because their linker bound to the target, so strong binders are not as distinguished. The amplification ability indicates the ability to survive the selection process to the next round, which is equivalent to the challenge of binding to the target protein and surviving physical washes that can remove kinetically weak binders.

However, as discussed herein, laboratory directed evolution can be augmented with machine-learning techniques to improve the activity and diversity of discovered hits, such as particular macrocycle genotypes of interest, which generally have extended binding sites, allowing for increased binding affinity and selectivity. The discovered hits may exhibit improved performance, such as improved capability of binding to an antigen, such as a viral antigen, a tumor antigen, and/or the like. In many directed evolution experiments, the main result may include DNA sequencing data that measures the frequency of competing biomolecules (e.g., macrocycles) in the evolving population of biomolecules. However, biomolecule frequency may not be an accurate indicator of biological activity. To discover hits, one may want to sort biomolecules by their biological activity. This poses an inference problem, i.e., inferring biological activities of biomolecules given their frequencies.

A molecule discovery system may be used to run the directed evolution. In a DNA-encoded library (DEL) setting, the molecule discovery system may translate a DNA sequence into a peptide, or small protein, using a known codon table. In one embodiment, the alphabet size of the DNA sequence may be 4 and the alphabet size of the small protein may be 20. Peptides are physically connected to their DNA sequences by a linker.

At each time step, round, or generation of directed evolution, the molecule discovery system may conduct activity-based selection. The plurality of enrichment rounds comprises at least three enrichment rounds, and at least one of the enrichment rounds was a control round where the population of biomolecules is analyzed without the presence of a target protein. A set of peptide-linker-DNA compounds, more than 10¹³-10¹⁴, are challenged to bind to target proteins immobilized on a solid support. The molecule discovery system 100 may perform washing steps to remove peptide-linker-DNA compounds with weak binding strength and extract survivors. In a single round of selection, candidates may be challenged to bind to a target, then a washing step may be performed which may remove some binders (though washing may be intended primarily to remove very weak binders or non-binders).

At each time step, round, or generation of directed evolution, there may be mutation and amplification. The DNA sequence, and thus peptide identity, of binding compounds may be determined by DNA sequencing. Peptide-linker-DNA compounds may be amplified by polymerase chain reaction (PCR), which is a multi-step process that doubles DNA molecules each step by duplication. In other words, the population of biomolecules may be amplified by polymerase chain reaction (PCR) in each of the plurality of enrichment rounds. PCR can introduce 1×10⁻⁵ mutations per nucleotide per step.

After directed evolution is applied to the population of biomolecules, the molecule discovery system described herein learns a deep-learning model for biomolecule fitness selection. As an example and not by way of limitation, the molecule discovery system may train a deep neural network that estimates molecule fitness, representing at least the biological activity of the molecules, and use the fitness to rank biomolecules (e.g., macrocycles) for selection. The molecule discovery system may establish a fitness inference problem given on-target and off-target time series DNA sequencing data. The molecule discovery system may utilize maximum likelihood solutions for the nonlinear dynamical system induced by fitness-based competition. The disclosed approach may learn from multiple time series rounds in a principled manner, in contrast to prior work focused on two-round enrichment prediction. By ranking molecules based on fitness, the molecule discovery system can identify low-frequency, high-fitness molecules that may otherwise be missed by conventional systems. The molecule discovery system may additionally predict on-target and off-target binding fitness accurately, discover novel and diverse genotypes of the biomolecules, and improve the quantity and diversity of identified molecules. The experiments show that inferring fitness while jointly learning a sequence-to-fitness deep-learning model (e.g., a transformer) improves performance over a baseline model without deep learning and a two-round enrichment baseline.

In particular embodiments, the molecule discovery system may access a biomolecule representation of a first biomolecule. The molecule discovery system may then process, by a machine-learning model, the biomolecule representation of the first biomolecule. The machine-learning model was trained using sequencing time-series data associated with biomolecule frequencies of particular biomolecules. The sequencing time-series data was obtained from a directed evolution of a population of biomolecules over a plurality of enrichment rounds. The population of biomolecules in each enrichment round may be a unique set of biomolecules with respect to each other enrichment round. As used herein, “unique” indicates that the population of biomolecules may have mutated in each enrichment round, thereby resulting in the population of biomolecules in each enrichment round having at least some unique biomolecules with respect to the population of biomolecules in each other enrichment round. The sequencing time-series data for each enrichment round may comprise a biomolecule frequency of each biomolecule of the population of biomolecules in the respective enrichment round. The training may comprise learning inferred fitness scores of the population of biomolecules for each enrichment round by predicting biomolecule frequencies of the population of biomolecules in the respective enrichment round given biomolecule frequencies of the population of biomolecules in one or more prior enrichment rounds. The molecule discovery system may further output, by the machine-learning model based on the processing of the biomolecule representation of the first biomolecule, an inferred fitness score for the first biomolecule.

### Automated System for Target-Binding Candidate Analysis

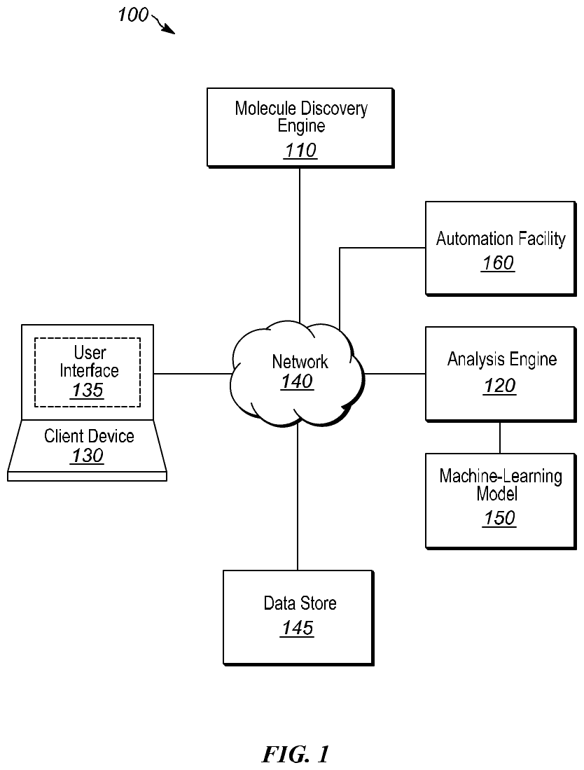

FIG. 1 depicts a system diagram illustrating an example of a molecule discovery system 100, in accordance with some example embodiments. Referring to FIG. 1, the molecule discovery system 100 may include a molecule discovery engine 110, an analysis engine 120, a client device 130, a data store 145, a machine-learning model 150, and an automation facility 160. As shown in FIG. 1, the molecule discovery engine 110, the analysis engine 120, the data store 145, the client device 130, and the automation facility 160 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like. The client device 130 may be a processor-based device including, for example, a workstation, a desktop computer, a laptop computer, a smartphone, a tablet computer, a wearable apparatus, and/or the like. The data store 145 may be a database including, for example, a relational database, a graph database, an in- memory database, a non-SQL (NoSQL) database, and/or the like. In some example embodiments, the molecule discovery engine 110 and/or the analysis engine 120 may be configured to support deep-learning based biomolecule fitness selection. In particular embodiments, the biomolecule can be a macrocycle.

In some example embodiments, the data store 145 may store data, such as a DNA-encoded library including a plurality of DNA sequences, which may be stored as peptides or small proteins. The molecule discovery engine 110 and/or the analysis engine 120 may perform, based on at least a portion of the data in the data store 145, an analytical workflow that includes applying a variety of computational analyses. The analysis engine 120 may, based on at least a portion of the data in the data store 145, train one or more machine-learning models, such as the machine-learning model 150, for downstream analytical tasks, and/or the like. For example, the results of the workflow may be used as training data to train a neural network (or another type of machine-learning model 150).

Referring again to FIG. 1, the molecule discovery engine 110 and/or the analysis engine 120 may perform the analytical workflow based on one or more user inputs received from the client device 130. For example, as shown in FIG. 1, the analysis engine 120 may generate, for display at the client device 130, a user interface 135. The one or more user inputs, which may be received via the user interface 135, may specify one or more subsets of data included in the data store 145. One or more visual representations of at least a portion of the results of the analysis performed by the molecule discovery engine 110 and/or the analysis engine 120 may be displayed as a part of the user interface 135. The user interface 135 may be interactive such that the types of the visual representations and the contents presented therein may be updated in response to the one or more user inputs.

In particular embodiments, the molecule discovery system 100 may utilize an automated analytical workflow to rapidly discover potent biomolecules. The automated analytical workflow may be performed by the automation facility 160. The automated analytical workflow may enable simultaneous visual observation and comparison of a large number of libraries for a round of selection for target binding and for round-to-round comparison. The automated analytical workflow can comprise receiving quantification information for a plurality of libraries of DNA-containing compositions. Receiving the quantification information may include collecting quantitative data for input molecules, positive molecules and negative molecules. The quantitative data may be generated after a round of automated library generation, target binding selection and DNA measurement by a quantitative method. The automated analytical workflow can then transfer quantification data and storing data in a database. Data transfer may include data scraping, in which a computer program extracts data from human-readable output coming from another program, e.g., Excel. The database stores each automatically export dataset and all associated metadata (e.g., date, time, and plate barcode, etc.) The automated analytical workflow can then assign datasets to a corresponding round of selection. Each exported dataset may be assigned to a corresponding round of selection by a user, in accordance with some embodiments. The automated analytical workflow can further visualize data on a graphical display surface. Once each dataset is assigned to a corresponding round, all the heatmaps and charts are generated according to pre-set criteria or a selection of filters.

More information on automated target binding analysis may be found in U.S. patent application Ser. No. 17/502,022, filed 14 Oct. 2021, particularly paragraphs 0065-0085, among other discussions in that patent application, the entirety of which is incorporated by reference.

### A Model of Evolutionary Dynamics

Challenges exist in how to select diverse biomolecules with improved binding capabilities from directed evolution experiments. One solution is to infer biomolecule fitness by parameterizing log relative fitness value per genotype based on sequencing time-series data obtained from directed evolution experiments and then select biomolecules based on the inferred fitness. Another solution is to utilize machine-learning techniques to infer biomolecule fitness based on sequencing time-series data obtained from directed evolution experiments and then select biomolecules based on the inferred fitness. In particular embodiments, the molecule discovery system 100 may use a model of evolutionary dynamics to handle the task of fitness inference. As an example and not by way of limitation, the molecule discovery system 100 may include a deep neural network that estimates biomolecule fitness, representing at least the biological activity of the biomolecules, and use the fitness to rank biomolecules (e.g., macrocycles) for selection.

Let G be the number of unique genotypes in a population. At time or round t, denote nₜ∈N^{G} as the vector of the number of each genotype, and Nₜ=Σᵢ₌₁^{G}n_{t,i} as the total count. This disclosure uses repeated subscripts for indexing: n_{t,i} is the count of the i-th genotype at time t. In population genetics, absolute fitness W∈( [symbol] ⁺)^{G} can be defined as

$${n}_{t + 1 , i} = {W}_{i} {n}_{t , i} . \qquad ( 1 )$$

In this disclosure, W describes the change in genotype counts due to differences in binding strength.

The molecule discovery system 100 infers absolute fitness from DNA sequencing time series data. The inferred fitness score for the first biomolecule may indicate a biological activity of the first biomolecule with respect to a target protein. The inferred fitness score for the first biomolecule may comprise an on-target fitness score associated the first biomolecule binding to a target protein. Denote mₜ as the count of sequencing reads, and Mₜ=Σᵢ₌₁m_{t,i} as the total read depth. The sequencing time-series data may comprise DNA sequencing time-series data, and the biomolecule frequencies of particular biomolecules may indicate genotype frequencies. DNA sequencing can be described with mₜ˜Multinomial (nₜ/Nₜ,Mₜ) so one can only estimate pₜ=nₜ/Nₜ, which are genotype frequencies. Without nₜ, one cannot infer W using equation (1). Equation (1) can be rewritten as (based on a proposition on relative fitness):

$${p}_{t + 1 , i} = \frac{{W}_{i}}{{\sum }_{j} {W}_{j} {p}_{t , j}} {p}_{t , i} . \qquad ( 2 )$$

In one embodiment, the proof for the proposition on relative fitness is as follows.

Using Nₜ₊₁=Σᵢn_{t+1,i}=ΣᵢWᵢn_{t,i}, we have:

$$\begin{matrix}{n}_{t + 1 , i} = {W}_{i} {n}_{t , i} & \\ \frac{{n}_{t + 1 , i}}{{N}_{t + 1}} = \frac{{W}_{i}}{{N}_{t + 1}} {n}_{t , i} & ( divided by {N}_{t + 1} ) \\ {p}_{t + 1 , i} = \frac{{W}_{i}}{{\sum }_{j} {W}_{j} {n}_{t} j} {n}_{t , i} & ( replace with definitions ) \\ {p}_{t + 1 , i} = \frac{{N}_{t} {W}_{i}}{{\sum }_{j} {W}_{j} {n}_{t , j}} \frac{{n}_{t , i}}{{N}_{t}} & ( multiply by {N}_{t} / {N}_{t} ) \\ {p}_{t + 1 , i} = \frac{{W}_{i}}{{\sum }_{j} {W}_{j} \frac{{n}_{t , j}}{{N}_{t}}} {p}_{t , i} & ( move {N}_{t} through fraction ) \\ {p}_{t + 1 , i} = \frac{{W}_{i}}{{\sum }_{j} {W}_{j} {p}_{t , j}} {p}_{t , i} & ( replace with definitions )\end{matrix}$$

Equation (2) indicates that W is identifiable, given pₜ, pₜ₊₁, only up to an unknown proportionality constant. Specifically, for any positive c, if equation (2) holds for some W, then it may also hold for absolute fitness values cW (based on a proposition on identifiability up to proportionality).

In one embodiment, the proposition on identifiability up to proportionality is as follows:

Suppose

$${p}_{t + 1 , i} = \frac{{W}_{i}}{{\sum }_{j} {W}_{j} {p}_{t , j}} {p}_{t , i} .$$

Then, for any positive scalar c,

$${p}_{t + 1 , i} = \frac{{cW}_{i}}{{\sum }_{j} {cW}_{j} {p}_{t , j}} {p}_{t , i} .$$

The proof for the proposition on identifiability up to proportionality is as follows:

$${p}_{t + 1 , i} = \frac{{W}_{i}}{{\sum }_{j} {W}_{j} {p}_{t , j}} {p}_{t , i} .$$

$${p}_{t + 1 , i} = \frac{{cW}_{i}}{c {\sum }_{j} {W}_{j} {p}_{t , j}} {p}_{t , i} .$$

$${p}_{t + 1 , i} = \frac{{cW}_{i}}{{\sum }_{j} {cW}_{j} {p}_{t , j}} {p}_{t , i} .$$

Due to this unidentifiability, the inference problem may be set up as: given mₜ for t=0, 1, . . . , T, infer relative fitness w=cW where c>0 is unknown.

Based on equation (2), the molecule discovery system 100 can simulate a non-linear dynamical system forward in time given an initial p₀ and relative fitness W. This simple model may ignore genetic drift and lack a mutational process. Ignoring genetic drift may be reasonable with 10¹⁰ population size, and PCR's low mutation rate may enable ignoring mutation effects for fitness inference purposes. Rewriting equation (2) using W in matrix notation, this disclosure defines predictions of a fitness model for round t+1 given previous round frequencies pₜ as

$${\hat{p}}_{t + 1} = \frac{w}{w \cdot {p}_{t}} ⊙ {p}_{t} \qquad ( 3 )$$

where ⊙ denotes pointwise multiplication. Based on equation (3), the molecule discovery system 100 may optimize W so that \hat{p}ₜ₊₁ reflects pₜ₊₁ accurately according to different noise models, thereby estimating fitness of biomolecules. Developing the model of evolutionary dynamics which optimizes the inferred fitness given the biomolecule frequency may be an effective solution for addressing the technical challenge of effectively establishing a fitness inference task for biomolecules undergoing mutations in enrichment rounds as such model accounts for the genetic drift and mutational process of the biomolecules in the enrichment rounds. Fitness may have the following properties. Fitness values cannot be negative. In addition, under strict mathematical assumptions, the W-bar (i.e., the multiplication of fitness and genotype proportion) may only increase over time under the model. The proportion of a genotype may increase if its fitness is above average and decrease if its fitness is below average. Increasing selection stringency may have the effect of increasing the variance or range of fitness. Increasing washing stringency or selection strength may help to better differentiate weak from strong binders. However, increasing washing too much may risk everything washing off (everything dying).

In practice, time-series data can be influenced not just by on-target binding strength but also off-target binding, where a molecule binds to the instrument instead of the target. In this case, the inferred fitness score for the first biomolecule may comprise an off-target fitness score associated the first biomolecule binding to a test instrument instead of the target protein. The molecule discovery system 100 can infer off-target fitness w_{off}∝W_{off} from off-target data: directed evolution time points where the target is not present, only the instrument, using equation (3). Furthermore, the molecule discovery system 100 can infer that wₜₒₜₐₗ∝Wₜₒₜₐₗ from standard directed evolution data, where both the target and the instrument are present. The molecule discovery system 100 infers Wₒₙ, on-target binding fitness, where it is assumed that Wₜₒₜₐₗ=Wₒₙ+W_{off}. Accounting for off-target fitness, inferring total fitness from standard directed evolution data, and then inferring on-target fitness may be an effective solution for addressing the technical challenge of handling the influence on the sequencing time-series data by both on-target binding strength and off-target binding as this approach may enable reasoning about the relative contributions of on-target and off-target fitness.

A challenge may include reconciling the unknown proportionality constants in these two fitness inference problems: in general, the scale of inferred W_{off} may be different, in an unknown manner, to the scale of Wₜₒₜₐₗ, which may be illustrated as follows:

$$\begin{matrix}\underbrace{{c}_{1} {W}_{total}} \\ available\end{matrix} = \begin{matrix}\underbrace{{c}_{1} {W}_{on}} \\ goal\end{matrix} + \begin{matrix}\underbrace{\frac{{c}_{1}}{{c}_{2}}} \\ unknown\end{matrix} \begin{matrix}\underbrace{{c}_{2} {W}_{off}} \\ available\end{matrix} . \qquad ( 4 )$$

In one embodiment, this disclosure discloses the following proposition for equation (4) on upper bound for relative off-target activity. Suppose

$$\begin{matrix}\underbrace{{c}_{1} {W}_{total}} \\ available\end{matrix} = \begin{matrix}\underbrace{{c}_{1} {W}_{on}} \\ goal\end{matrix} + \begin{matrix}\underbrace{\frac{{c}_{1}}{{c}_{2}}} \\ unknown\end{matrix} \begin{matrix}\underbrace{{c}_{2} {W}_{off}} \\ available\end{matrix} .$$

### Then

$$\frac{min {w}_{total}}{max {w}_{off}} = \frac{min {c}_{1} {W}_{total}}{max {c}_{2} {W}_{off}} \geq \frac{{c}_{1}}{{c}_{2}} .$$

The proof for the above proposition is as follows.

Using the property that absolute fitness is non-negative,

$${c}_{1} {W}_{total} = {c}_{1} {W}_{on} + \frac{{c}_{1}}{{c}_{2}} {c}_{2} {W}_{off}$$

$${c}_{1} {W}_{total} \geq 0 + \frac{{c}_{1}}{{c}_{2}} {c}_{2} {W}_{off}$$

$$\frac{{c}_{1} {w}_{total}}{{c}_{2} {w}_{off}} \geq \frac{{c}_{1}}{{c}_{2}}$$

This relationship holds for each entry in the vector.

As can be seen, this disclosure proves an upper bound for c₁/c₂, which enables reasoning about the relative contributions of on-target and off-target fitness:

$$\frac{min {w}_{total}}{max {w}_{off}} > \frac{{c}_{1}}{{c}_{2}} . \qquad ( 5 )$$

Alternatively, the molecule discovery system 100 may learn a scale for Wₒₙ relative to W_{off} by fitting to the data. This disclosure uses this approach for the experiments. Although this disclosure describes the model of evolutionary dynamics including particular covariates, this disclosure contemplates that the model can be easily extended to include additional covariates, if present.

Many conventional approaches learn enrichment scores, which may be equivalent to relative fitness with two time points, but not with more than 2 time points (i.e., a proposition on enrichment being not equivalent to relative fitness with % 2 time points). This proposition may be illustrated as follows. Suppose

$${p}_{t + 1 , i} = \frac{{W}_{i}}{{\sum }_{j} {W}_{j} {p}_{t , j}} {p}_{t , i}$$

for some fitness W holds for p₀≠p₁≠p₂. Then, enrichments p₂/p₁≠p₁/p₀. The proof is as follows.

Rearranging the relative fitness equation, each enrichment is equal to a fraction with W in the numerator, and ΣⱼWⱼp_{t,j} in the denominator. The numerator is the same for p₂/p₁, p₁/p₀, but the denominator is not the same. So, the enrichments are not equal.

Enrichment is equivalent to relative fitness with 2 time points. Specifically, algebraic rewriting of equation (2) shows that enrichment is proportional to absolute fitness with 2 time points. Relative fitness is also proportional to absolute fitness.

When there are more than 2 time points where each pair of time points are consistent with the same W, computing enrichment may yield different enrichment values for each pair of time points. Thus, enrichment cannot be equivalent to relative fitness.

In contrast to the prior works, the embodiments disclosed herein present a principled method for datasets with more than 2 time points. For hit prioritization, biologists may rank compounds by frequency in the last time point. However, the motto is “survival of the fittest”, not “survival of the most frequent.” In other words, ranking by fitness may identify low-frequency, high-fitness “rising stars” otherwise missed, particularly by ranking by frequency alone.

### Methodology

With the model of evolutionary dynamics established, the molecule discovery system 100 may perform fitness inference on biomolecules for biomolecule selection. Fitness inference can be split into two components. One component may include a differentiable parameterization of W to map from genotype to fitness. Another component may include a differentiable loss function used to optimize how well the predicted frequencies, \hat{p}ₜ₊₁ from equation (3), match the observed frequencies pₜ₊₁. Given these two components, fitness may be inferred using first order optimization.

In particular embodiments, fitness inference may be based on different loss functions. One example loss function may be multinomial negative log-likelihood loss. A simple approach to learning fitness may be to take the predicted frequencies from equation (3) and treat them as the event probabilities in a multinomial distribution. Then the log-likelihood of a round of read counts given the fitness may be:

$$log p ( {m}_{t + 1} \vert {p}_{t} , w ) = log Mult [ p = {\hat{p}}_{t + 1} , C = {M}_{t + 1} ] ( {m}_{t + 1} ) \qquad ( 6 )$$

where Mult[p,C](mₜ₊₁) is the multinomial likelihood of mₜ₊₁ with event probabilities p and event count C.

Another example loss function may be Dirichlet-multinomial negative log-likelihood loss. In particular embodiments, learning the inferred fitness scores in the training of the machine-learning model may comprise optimizing a Dirichlet-multinomial loss function. The Dirichlet-multinomial loss function utilizes an over-dispersed multinomial distribution to account for an increased difficulty associated with predicting biomolecule frequencies of the population of biomolecules in each enrichment round given biomolecule frequencies of the population of biomolecules in a prior enrichment round. The training of the machine-learning model may further comprise calculating a Dirichlet loss negative log-likelihood between the predicted biomolecule frequencies and actual biomolecule frequencies as a negative log-likelihood.

By construction, relative fitness, i.e., equation (2), may account for the frequencies and not the read counts at round t. However, in practice, the intrinsic high-assay noise may make the measured enrichment less reliable for lower counts. The multinomial distribution previously described may not account for prior round counts, preventing modeling of this aspect. Therefore, the disclosure introduces an over-dispersed multinomial distribution, the Dirichlet-multinomial (DM), to account for the increased difficulty (i.e., lower confidence) of predicting pₜ₊₁ from pₜ when the total read counts are lower. Using a Dirichlet-multinomial loss to optimize the fitness inference task may be an effective solution for addressing the technical challenge of the measured enrichment being less reliable for lower counts due to high assay noise as the Dirichlet-multinomial loss may account for the increased difficulty of predicting biomolecule frequency at a current round from a prior round when the total read counts are lower.

The DM distribution is the posterior predictive distribution of a Dirichlet prior on a multinomial model of count data. When updating a Dirichlet prior with concentration α with observed counts per category k, the posterior is Dirichlet distributed with concentration α+k. The total concentration may increase from Σᵢαᵢ to Σᵢ(αᵢ+kᵢ). Based on this observation, this disclosure considers a DM likelihood α=Mₜ\hat{p}ₜ₊₁, with \hat{p}ₜ₊₁ computed as in equation (3), and Mₜ being the total read counts. Note that the expected frequency of this distribution is \hat{p}ₜ₊₁ (as in the multinomial distribution), but the total concentration is Mₜ, thus accounting for the total counts. This yields the log-likelihood for mₜ₊₁:

$$log p ( {m}_{t + 1} \vert {m}_{t} , w ) = log D M [ α = {M}_{t {\hat{p}}_{t + 1}} , C = {M}_{t + 1} ] ( {m}_{t + 1} ) \qquad ( 7 )$$

where DM[α,C](mₜ₊₁) is the Dirichlet-multinomial likelihood of counts mₜ₊₁ with concentration parameters α and total count C. The DM loss may be differentiably calculated using Pyro [7] as the negative DM log-likelihood.

The molecule discovery system 100 may utilize a variety of approaches for parameterizing fitness. One approach may include defining one log relative fitness value per genotype, which is referred per-genotype fitness in this disclosure. Another approach may include using a neural network to map from genotype to log relative fitness, which is referred deep-learning fitness model in this disclosure.

In e, one log w parameter may be trained for each genotype. The molecule discovery system 100 may randomly initialize the log fitness values using a standard normal distribution. Per-genotype fitness may have the advantage of training in a few seconds on GPU and having relatively few hyperparameters to tune. It may have the disadvantages of being unable to make predictions on genotypes on which it was not trained and not taking advantage of the fact that similar genotypes likely have similar fitness. As an example and not by way of limitation, the L-BFGS algorithm was found to be effective for training per-genotype fitness. The molecule discovery system 100 may select the learning rate for each loss function using grid search.

In deep-learning fitness model, a neural network model may parameterize the mapping from genotype to log w. In other words, the machine-learning model may comprise one or more neural networks. The model may learn general genotype motifs described in the latent space, which are linked to high/low fitness values. Therefore, the model may leverage the inductive bias that enrichment (and in general, fitness) is a function of the genotype. Consequently, this parametrization may allow extrapolating fitness on new genotypes not observed in the directed evolution experiment. This may enable further virtual screening, discovery of high fitness motifs, and in-silico optimization. As a result, the embodiments disclosed herein may have a technical advantage of identifying the fitness of biomolecules not in the original enrichment rounds as the molecule discover system 100 utilizes a deep learning model that can infer fitness for any unseen biomolecules.

In particular embodiments, the molecule discovery system 100 may generate the biomolecule representation of the first biomolecule which may be a polypeptide corresponding to a first genotype. The generating may comprise determining a plurality of amino acids of the first biomolecule, applying a function to determine a feature representation for the respective amino acid for each amino acid of the plurality of amino acids, and generating a genotype representation corresponding to the first genotype based on the plurality of feature representations associated with the plurality of amino acids. To represent the genotypes, instead of one-hot encoding the amino acids (aⱼ), the molecule discovery system 100 may embed the structure of each aⱼ through its chemical descriptor MF(aⱼ), where MF is the hashed Morgan fingerprint function with substructure counts. By doing this, (1) the model may learn generalizable amino acid features, and (2) the system may inject an inductive bias in the aⱼ space. Applying MF to genotype gi may yield the sequence of tokens xᵢ=[MF(a_{i,1}), MF(a_{i,2})], . . . , MF(a_{i,L})].

In one example embodiment, the molecule discovery system 100 used a transformer-like encoder [9] with multi-head attention to map from the embedded amino acid sequences to log w. The transformer-like encoder may have 8-head multi-head attention, 64-dimensional feedforward layers, and a single linear layer at the end to predict fitness. The models were trained for 500 epochs of 25,600 samples using the AdamW optimizer with grid search per loss function selected cosine annealed learning rate, batch size, and weight decay.

The molecule discovery system 100 may optimize hyperparameters for fitness inference models with grid search. In particular embodiments, the one or more neural networks may comprise a first neural network trained for predicting on-target fitness scores associated with biomolecules and a second neural network trained for predicting off-target fitness scores associated with biomolecules. For on-target and off-target fitness models, the molecule discovery system 100 may first optimize the off-target model, and then the on-target model using the frozen off-target model previously optimized. As an example and not by way of limitation, the following grid of hyperparameters may be considered. The learning rate may be [10⁻⁴, 10⁻³, . . . , 10⁻²]. The weight decay may be [0, 10⁻⁴, 10^{-3.5}, 10⁻³, . . . , 10⁻¹]. The batch size may be [64, 128, 256]. The weights from each training run may be taken using early stopping on the validation loss. In one example embodiment, ten percent of training data may be randomly selected as validation data for model selection.

In one embodiment, to learn the unsupervised genotype space, the molecule discovery system 100 trained a VAE using the same encoder and featurization described for the deep-learning fitness model. A fully connected decoder reconstructs both the peptide sequence and the embedding of each amino acid, thus learning amino acid similarity on top of sequence similarity. The final loss is therefore the sum of the KL-divergence, the sequence reconstruction, and the amino acid reconstruction. Final representations are obtained from the latent space and shown after 2D projection with UMAP.

Equation (3) may only enable predictions between two consecutive rounds, so each minibatch may only contain two consecutive rounds with the same subset of genotypes. To ensure the loss is defined for each batch, the molecule discovery system 100 may use a simple batch sampling algorithm. The pseudo-code of this algorithm is shown below.

```
Def sample_batch (
    batch_size: int,
  genotypes: np. Ndarray, # G x genotype embedding dimension
  count_matrix: np. Ndarray, # G x T
) −> tuple(np. Ndarray, np. Ndarray): # X, y
  T = count_matrix. Shape [1]
  t = np. Random. Randint (0, T − 1)
  log_enrichment_defined_mask = (
    (count_matrix[:, t] > 0) & (count_matrix[:, t + 1] > 0)
  )
  log_enrichment_defined_idx = np. Where (
    log_enrichment_defined_mask,
  )[0]
  batch_idx = np. Random. Choice (
      log_enrichment_defined_idx,
      batch_size,
  )
  X = genotypes[ batch_idx]
  y = count_matrix[batch_idx, [t, t + 1]]
  return X, y
```

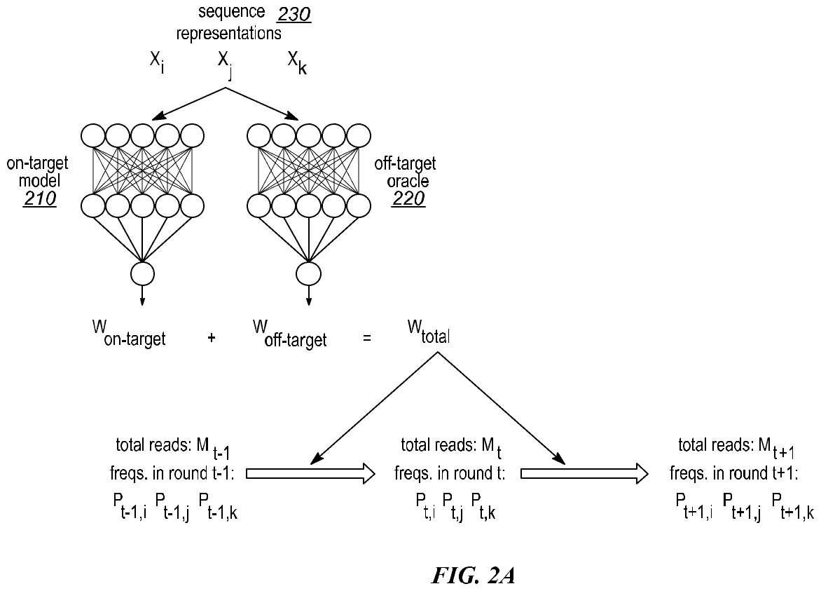

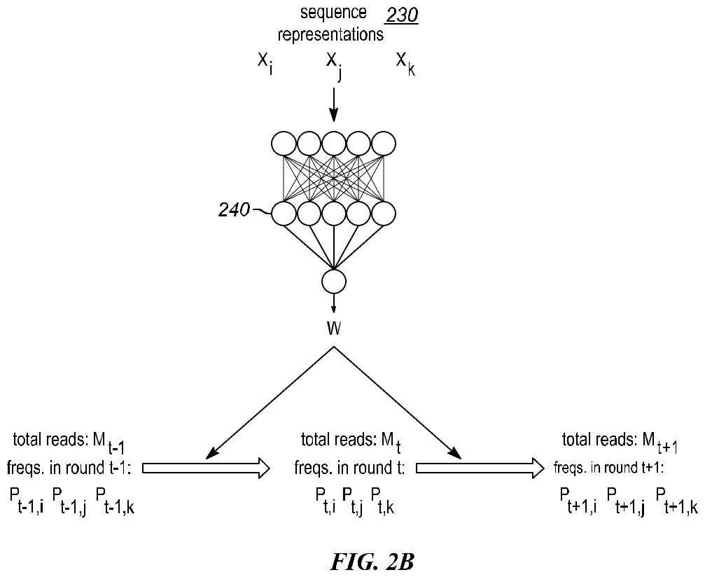

FIG. 2A illustrates an example deep-learning fitness model with off-target oracle. On-target neural network (i.e., on-target model 210) and off-target neural network (i.e., off-target oracle 220) may predict fitness from genotype (represented by the sequence representations 230), which can be used to predict future round genotype frequencies. As previously described, the molecule discovery system 100 may separate total fitness Wₜₒₜₐₗ into an on-target and (one or more) off-target contributions. W_{off} may be inferred using the off-target rounds, and Wₒₙ using the all-target rounds through equation (4), with any combination of fitness parameterization and loss function. In other words, the inferred fitness score for the first biomolecule may comprise an on-target fitness score associated the first biomolecule binding to a target protein and an off-target fitness score associated the first biomolecule binding to a test instrument instead of the target protein. By inferring the fitness contributions separately, the molecule discovery system 100 may select genotypes with better specificity. The molecule discovery system 100 may determine a binding specificity of the first biomolecule based on a ratio of the on-target fitness score to the off-target fitness score. As used herein, “binding specificity” indicates the binding pattern of the first biomolecule with respect to the target protein and the test instrument. For example, low binding specificity indicates the first biomolecule binding more strongly to the test instrument whereas high binding specificity indicates the first biomolecule binding more strongly to the target protein. As a result, the embodiments disclosed herein may have a technical advantage of the ability to filter out low specificity binders from the results of the selection as the molecule discovery system 100 may determine both on-target and off-target bindings.

FIG. 2B illustrates another example deep-learning fitness model. A neural network 240 may predict fitness from genotype (represented by the sequence representations 230) without separating total fitness into an on-target and (one or more) off-target contributions. The predicted fitness W can be used to predict future round genotype frequencies.

The training of the machine-learning model may further comprise pretraining an off-target model. In practice, the molecule discovery system 100 may first pretrain an off-target fitness model on the off-target rounds only. The pretraining may comprise identifying one or more off-target enrichment rounds from the plurality of enrichment rounds, and pretraining the off-target model based on sequencing time-series data for the one or more off-target enrichment rounds. This may provide an effective solution, as (1) the molecule discovery system 100 may leverage off-target data measured for different targets, thus potentially increasing the data size, and (2) the molecule discovery system 100 may efficiently re-use the pretrained model for new targets. The pretrained “off-target oracle” may be then frozen and the on-target model may be trained using the all-target rounds through equation (4). In other words, the training of the machine-learning model may further comprise accessing sequencing time-series data from one or more on-target enrichment rounds from the plurality of enrichment rounds and generating an on-target model based on the accessed sequencing time-series data from the one or more on-target enrichment rounds and the off-target model. The embodiments disclosed herein compared this approach to jointly learning on-and off-target fitness and observed that the latter results in less stable training and significantly longer training time.

### EXAMPLES

The presently disclosed subject matter provides for improved biomolecule selection based on fitness. The following describes the examples for fitness inference and biomolecule selection.

### Example 1: Directed Evolution Data

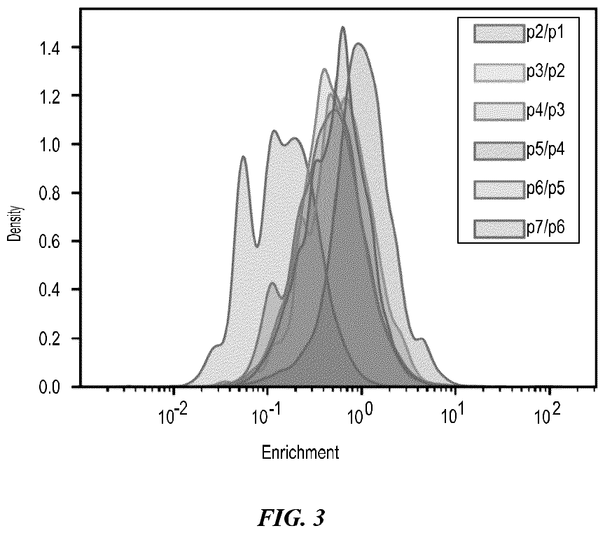

The directed evolution experiment analyzed in the following includes seven rounds (the first round is the random library), and three off-target rounds following rounds five, six and seven. The directed evolution experiment selects for activity for the target, and the library includes 8-mers (cyclic peptides). The embodiments disclosed herein split the data by genotype into a training and a test set, and holdout round seven and off-target seven to use as holdout rounds. The embodiments disclosed herein then filtered the training and test sets so that each genotype had a count of at least 10 in one on-target round and appeared in at least two consecutive rounds. The final number of training/test genotypes is summarized in Table 1. FIG. 3 illustrates an example distribution of the enrichment for each pair of rounds. More specifically, FIG. 3 shows kernel density plot of log enrichment ratios of the filtered genotypes by pair of time points.

TABLE 1
Final number of training/test genotypes

| Cohort | M₁ | M₂ | M₃ | M₄ | M₅ | M₆ | M₇ |
|---|---|---|---|---|---|---|---|
| train | 2180 | 14765 | 17419 | 18233 | 18476 | 16844 | — |
| test | 233 | 1598 | 1926 | 2025 | 2054 | 1878 | 1673 |
| train (off-target) | — | — | — | — | 15285 | 11834 | — |
| test (off target) | — | — | — | — | 1675 | 1300 | 375 |

### Example 2: Fitness Inference Comparison with Baselines

The embodiments disclosed herein compared the disclosed fitness inference approach to three baselines. Prior round frequency assumes that enrichment is equal to the frequency in the final time point of the training data—in other words, the most fit genotypes are the most frequent. Prior round enrichment assumes that the holdout round's enrichment is equal to the penultimate round's enrichment. Enrichment regression introduces a regression loss function that can be used with any of the fitness parameterizations. The loss is the mean squared error between the predicted and actual log-enrichment for every pair of rounds. Enrichment is defined as the ratio of genotype frequencies in a round and the prior round, pₜ/pₜ₋₁. This baseline may correspond to naïvely extending enrichment regression to data with multiple time points. It may not account for the fact that enrichment depends not only on a genotype, but also on the fitness of competing genotypes in the same selection round.

The embodiments disclosed herein evaluated model performance through two generalization tasks. First, the embodiments disclosed herein evaluated all models' abilities to predict the enrichment of the holdout final round T trained on time points 1, . . . , T−1, for the training sequences (Table 2, Seen Molecules). In this case, the first biomolecule is within the population of biomolecules in the plurality of enrichment rounds. Second, the embodiments disclosed herein evaluated the ability of the deep-learning fitness model to predict the enrichment in round T on holdout genotypes (Table 2, Unseen Molecules). In this case, the first biomolecule is not within the population of biomolecules in the plurality of enrichment rounds. In particular embodiments, the molecule discovery system 100 may determine, based on the inferred fitness score for the first biomolecule, whether a biological activity associated with the first biomolecule meets a predetermined criteria for selection. Pearson-r is reported to measure the agreement between actual and predicted enrichment. Pearson-r weights the high enrichment sequences most heavily, thus reflecting the goal of selecting high fitness genotypes. When an off-target oracle is used, the individual contributions (on-target and off-target fitness) are aggregated for the evaluation.

TABLE 2
Enrichment prediction Pearson correlation I for seen
and unseen molecules in holdout final round (higher
is better). Bold indicates the difference from the
best results were not statistically significant.

| | r Seen | r Unseen |
|---|---|---|
| Parameterization | molecules (↑) | molecules (↑) |
| Prior round frequency | 0.03 | — |
| Prior round enrichment | 0.29 | — |
| Per-genotype (Enrichment regression) | 0.15 ± 0.07 | — |
| Per-genotype (Multinominal) | 0.34 ± 0.13 | — |
| Per-genotype (DM) | 0.38 ± 0.02 | — |
| Deep-learning fitness (Enrichment | 0.25 ± 0.05 | 0.22 ± 0.06 |
| regression) | | |
| Deep-learning fitness (Multinominal) | 0.16 ± 0.02 | 0.10 ± 0.03 |
| Deep-learning fitness (DM) | 0.40 ± 0.02 | 0.45 ± 0.07 |
| Deep-learning fitness (DM, Off-target | 0.39 ± 0.07 | 0.38 ± 0.16 |
| oracle) | | |

The results show the benefits of using the fitness inference framework compared to all the baselines. The prior-round frequency and enrichment baselines predicted enrichment poorly compared to the disclosed fitness-based methods, demonstrating that enrichment is driven by the most fit accounting for all rounds, not just the last round. Fitness inference with the DM loss also outperformed enrichment regression when using either parameterization, which shows the benefits of probabilistic losses built on the fitness framework. Between the two fitness-based losses, the DM loss yielded improved results over the multinomial loss, which struggled with the deep-learning fitness model parameterization. This may indicate that accounting for prior round read counts improves model performance. As can be seen, the embodiments disclosed herein may have a technical advantage of more effective selection of discovered hits as the molecule discovery system 100 infers fitness of biomolecules that indicates biological activity and then determines discovered hits based on such biological activity.

### Example 3: On-Target and Off-Target Fitness Inference

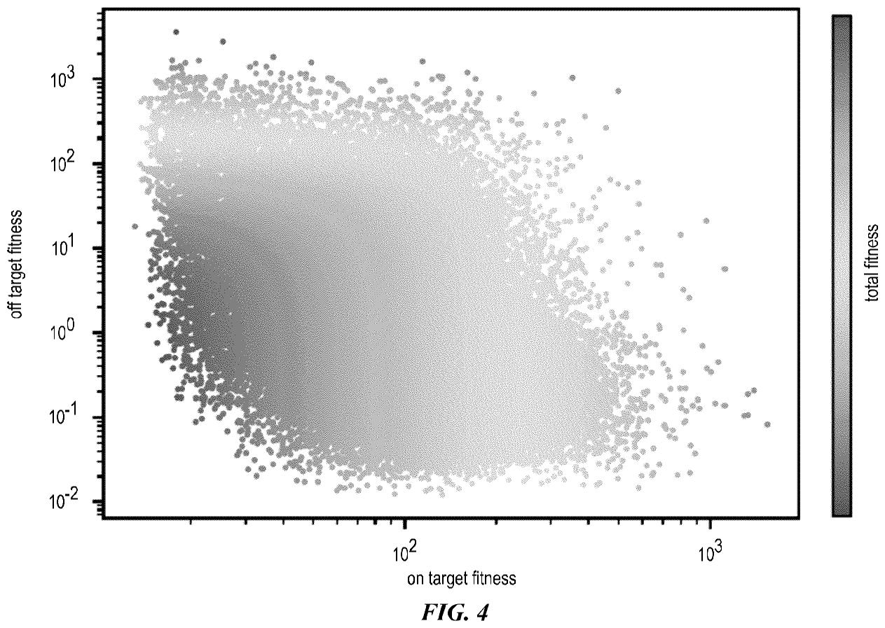

The embodiments disclosed herein analyzed the individual fitness contributions Wₒₙ and W_{off} training the model with off-target and all-target rounds. FIG. 4 illustrates example on-target fitness versus off-target fitness regarding predictions on 100,000 holdout genotypes. The grayscale intensity corresponds to total fitness. As shown, the same total fitness corresponds to different on-target/off-target ratios. The dataset is characterized by high off-target binding, which translates into an overall high off-target fitness. Thus, the pipeline in this disclosure may be used to filter out low specificity binders from the results of the selection. Retraining the model, the embodiments disclosed herein observed a high consistency for the inferred on-target/off-target fitness ratios (pairwise Spearman correlation=0.66, three replicates).

The embodiments disclosed herein assessed the usefulness of the estimated fitness to highlight novel and diverse genotypes not easily detected through the standard last-round frequency pipeline, wherein the genotypes with high frequency in the last selection round are considered to be the most fit ([12; 13; 14; 15; 16; 17; 18]). In particular embodiments, the molecule discovery system 100 may process a plurality of biomolecule representations associated with a plurality of respective biomolecules by the machine-learning model to determine a plurality of inferred fitness scores for the plurality of biomolecules, respectively. The molecule discovery system 100 may further select, based on the inferred fitness scores for the plurality of biomolecules, one or more biomolecules meeting a predetermined criteria for selection. One or more of the selected biomolecules may be each associated with a low relative biomolecule frequency in a last round of the plurality of enrichment rounds.

### Example 4: Discovery of Novel and Diverse Genotypes

In particular embodiments, the molecule discovery system 100 may select a diverse set of biomolecules as discovered hits based on the fitness scores. The biomolecules in the diverse set of biomolecules may meet a predetermined criteria for selection. The molecule discovery system 100 may calculate distance metrics between the biomolecules. As an example and not by way of limitation, the distance metric may be based on edit distance. As another example and not by way of limitation, the distance metric may be based on a distance determined based on a method that identifies distinct peptides. More information on identifying distinct peptides may be found in U.S. patent application Ser. No. 18/338,772, filed 21 Jun. 2023, particularly paragraphs 0069-0120, among other discussions in that patent application, the entirety of which is incorporated by reference. As yet another example and not by way of limitation, the distance metric may be based on VAE (variational auto encoder). In one embodiment, the training of the VAE may be performed with reconstruction loss and monomer prediction. For further clarity, the VAE may be trained to reconstruct the biomolecule that was input. The embedding layer at the bottleneck may allow for generating embeddings of biomolecules by inserting biomolecules into the trained VAE and reading out the vector at the embedding layer at the bottleneck of the VAE. Consequently, the distance between two biomolecules can be calculated based on the Euclidean distance between two vectors for the two biomolecules generated this way. The molecule discovery system 100 may then cluster the biomolecules based on the distance metric. The molecule discovery system 100 may further select the diverse set of biomolecules based on both the fitness scores and the clusters (e.g., selecting biomolecules with high fitness scores from diverse clusters). In other words, the predetermined criteria may be based on both the fitness scores and the clusters. As an example and not by way of limitation, the molecule discovery system 100 may use an algorithm to select the diverse set of biomolecules. The algorithm may be heuristic, mixing high fitness scores and diverse clusters based on its assessment. For example, the algorithm may select biomolecules from two or more diverse clusters.

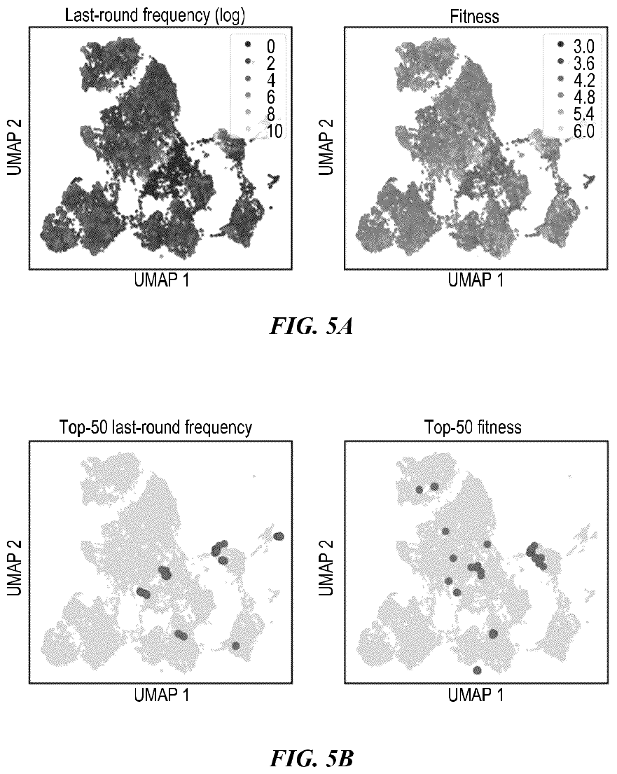

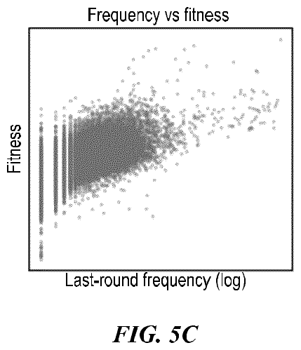

The molecule discovery system 100 may further generate a genotype space based on the biomolecule frequencies and the inferred fitness scores for the plurality of biomolecules. The molecule discovery system 100 may then select the one or more biomolecules by identifying the one or more biomolecules from one or more regions in the genotype space. Each of the one or more regions may be associated with a particular biomolecule frequency range and a particular biomolecule fitness range. FIG. 5A illustrates an example genotype space generated based on last-round frequency and last-round frequency winners. The grayscale intensity corresponds to log-scaled frequency and fitness, respectively. FIG. 5B illustrates an example genotype space generated based on fitness and fitness winners. FIG. 5C illustrates an example last-round frequency versus fitness. The molecule discovery system 100 embedded genotypes in a meaningful “genotype space” learned in an unsupervised fashion through a VAE and embedded with UMAP and compared the high-frequency and high-fitness regions in this space. (FIG. 5A). As shown, while several areas share high frequency and high fitness values, one may also identify regions of genotypes characterized by high fitness but low frequency (and vice-versa). Such regions may help improve the number and diversity of the identified biomolecules. Focusing on the top-50 “winners” through the two criteria (FIG. 5B), one may observe that the inferred fitness helps selecting genotypes distant (i.e., less similar) to those selected through frequency. Indeed, analyzing the agreement between last-round frequency and fitness, one may notice that, especially for low-frequency values, the estimated fitness spans a broad frequency range (FIG. 5C). As can be seen, the embodiments disclosed herein may have a technical advantage of the ability to discover novel and diverse genotypes as the molecule discovery system 100 may identify genotypes with high fitness but low frequency (and vice versa), which may be missed through standard approaches for biomolecule discovery.

### Example 5: Efficiency of Hits Discovery

The embodiments disclosed herein may identify hits months earlier than traditional methods. Inferring fitness can identify hits multiple timepoints earlier. For example, in one example case, each timepoint was up to 1 month apart, meaning one could potentially identify hits more than 2 or 3 months earlier than the baseline.

### Example 6: Identification of Short Macrocycles

The embodiments disclosed herein may additionally identify short macrocycle hits without dedicated short macrocycle libraries. In a traditional protocol, dedicated short macrocycle libraries may be not often run because historically they may not yield any good results. Fitness inference with sufficiently deep sequencing depth may enable identifying short macrocycles (6-9 aa) in a general macrocycle library (6-14 aa) even though long macrocycles (10-14 aa) dominate in frequency.

### Additional Denoising

As described earlier, to discover hits, biomolecules undergo multiple rounds of selection involving iterative steps of incubation with the target protein, washing off non-binders, amplification, and re-translating DNA sequences to macrocycle peptides. The embodiments disclosed herein considered the washing steps when modeling the absolute fitness, or the number of children, of each individual genotype. One may assume that each genotype has a probability of binding, and then a probability of surviving the washing. Binding probabilities may be exponentially distributed, with very few hits having high binding probability and most genotypes having near zero probability. Since binders are rare, one may imagine the washing as a bottleneck that funnels a large number of candidate genotypes (e.g., 1013 genotypes) down to a much smaller number, e.g., 107 genotypes (indicating 1 in a million chance of binding, averaged over all genotypes). For washing, it may be reasonable to assume that each genotype's likelihood of surviving the wash is independent, which means it follows a binomial distribution. By the properties of the binomial distribution, washing may not change fitness estimates, but it may add binomial sampling noise, especially when the number of copies of a genotype that bound is low. Short macrocycles may be weak binders, so they may leave very small number of copies of a genotype that bound after binding. The washing step may then introduce binomial noise on small number of copies of a genotype that bound.

Binomial sampling may introduce noise into genotype frequencies over time as follows. Between each selection step, aliquots may be taken, and samples may be also taken for DNA sequencing. As an example and not by way of limitation, frequency counts may be convoluted by Ribosomal/translation biases and selection artifacts. The molecule discovery system 100 may utilize a probabilistic model of observations to compute a p-value, or Bayes factor, for genotype activity to determine how much of its time series trajectory is due to random noise versus fitness-based selection. The strength of evidence can be evaluated using a model of baseline to generate p-values, false discovery rate, or Bayes factor. For example, the probability of observing three, rather than just one, sequences with timeline trajectories suggestive of high fitness, may be lower under a noise model.

In an example case, Ribosomal/translation biases may complicate fitness inference since sequences that ribosomes translate more efficiently effectively may have more “children” each generation. In particular embodiments, the molecule discovery system 100 may handle Ribosomal/translation biases by incorporating a simple model of ribosome translation efficiency into deep-learning fitness model. As an example and not by way of limitation, a method may be to include it as a log additive term. Let r be the ribosome translation efficiency of a genotype, scaled such that if r of some genotype is 2× the r value of another genotype, then the first genotype has twice as many translated products. Then the translation-controlled fitness f may be inferred according to data such that fr=w, where w is the effective fitness that governs population changes. A simple model of ribosome translation efficiency may be, for instance, the average codon score in a sequence, since ribosome efficiency may have a bias by codon.

The deep-learning fitness model can better distinguish signal from noise, thereby identifying short macrocycles with meaningful activity. The deep-learning fitness model may leverage more information, i.e., aggregating across all timepoints and using a probabilistic model of observations and noise. The deep-learning fitness model may be used to infer the fitness of each peptide by aggregating information across all timepoints under a probabilistic model. The deep-learning fitness model may be able to control for ribosomal translation efficiency. Furthermore, the molecule discovery system 100 may increase power by aggregating information across peptides within the same family.

In particular embodiments, the deep-learning fitness model may further incorporate family clustering. With the access to a pairwise amino acid similarity matrix, defined using chemical atom-atom-path similarity and expert knowledge, the molecule discovery system 100 may utilize a similarity function between two sequences as the average pairwise similarity in their amino acids. A first step may be to cluster genotypes by similarity and report fitness statistics for the cluster: max, median, mean, number of genotypes in cluster, cluster similarity, etc. Family clustering may strengthen the evidence for individual hits, if there are highly related genotypes with trajectories that also support high fitness estimates. The similarity metric may be also used to generate unseen sequences from a seed input sequence. While experimentalists may do this manually, they may find a computational algorithm useful to easily design libraries at scale. As an example and not by way of limitation, an algorithm may be to generate all mutants within a predetermined hamming distance from the seed, discard them if they were observed in the data already, rank the rest by similarity, and return them.

In one embodiment, the molecule discovery system 100 may utilize a model for sampling macrocycles and interpreting families. Descending from the root to a leaf of a hierarchical clustering tree may be viewed as a denoising procedure, where one may start from something general and gradually add specificity. However, while a single hierarchical clustering is a tree, there can be multiple valid ways to cluster. Moreover, clustering may typically maximize the sequence similarity between child nodes, but in certain embodiments, it may be ok with clusters that are slightly more different in sequence but have more similarity in fitness. In terms of macrocycles, there may be multiple families that a single macrocycle peptide can belong to. Clustering may only assign individuals to a single family. The model for sampling macrocycles and interpreting families, in contrast, may handle relationships better described as Directed Acyclic Graphs (DAGs). As an example and not by way of limitation, the initial state of the model may be a maximally generic macrocycle comprising amino acids. At each state, the set of available actions may transform some abstract symbol into more specific symbols: e.g., any amino acid can be transformed into a symbol representing the set of amino acids that are hydrophobic, polar, or positively/negatively charged. The final states may be the observed macrocycle peptide sequences. In particular embodiments, the model for sampling macrocycles and interpreting families may be trained with r(x)=fitness, and once trained, may be able to sample new macrocycles based on the fitness distribution, benefiting from all the generalization power and potential composability of the model. To interpret clusters, one may analyze and compare the flow of intermediate states (which represent macrocycle families).

### Discussion

This disclosure introduced fitness inference for directed evolution time-series data with on-target and off-target selection rounds. The embodiments disclosed herein developed and compared two parameterizations of fitness, i.e., per-genotype and deep-learning fitness model, with two different probabilistic loss functions. With both parameterizations, fitness inference with the DM loss significantly improved results compared to baselines that do not account for the competition induced in a multi-round selection experiment. Compared to the per-genotype parameterization, deep-learning fitness model enabled fitness prediction on novel-unseen genotypes learning a general “motifs to fitness” mapping, with the assumption that similar genotypes result in similar fitness values.

This disclosure demonstrated the ability of the fitness framework to elegantly include off-target binding data. Compared to regression modeling, the disclosed fitness framework may not require learning explicit trade-off parameters for off-target binding. It is observed that the on-target-and-off-target strategy slightly negatively impacted performance with respect to directly learning total fitness. This may have been caused by the model requiring twice the number of parameters and may be improved exploring weight sharing and other regularization techniques. However, the performance impact may be worth the ability to disentangle total fitness into its contributions for downstream filtering.

Finally, this disclosure assessed the diversity of the genotypes with the highest fitness (estimated by deep-learning fitness model with the DM loss) compared to those with the highest last-round frequency. It is found that, while the fitness-based selection rediscovers genotypes similar to those selected solely through frequency, it also highlights genotypes in unexplored regions, thus improving the overall diversity.

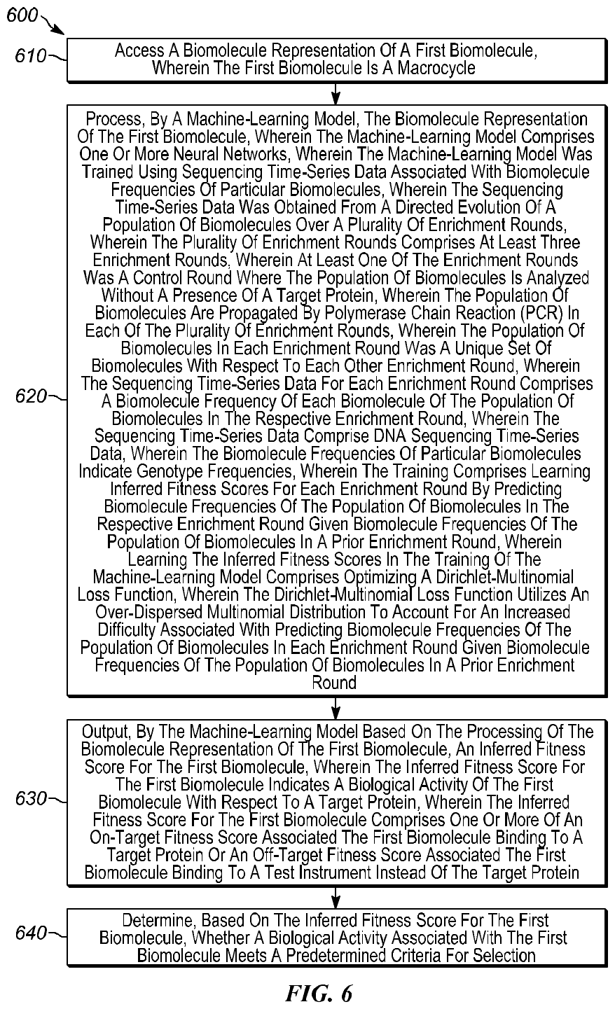

FIG. 6 illustrates an example method 600 for biomolecule fitness inference. The method may begin at step 610, where the molecule discovery system 100 may access a biomolecule representation of a first biomolecule, wherein the first biomolecule is a macrocycle. At step 620, the molecule discovery system 100 may process, by a machine-learning model, the biomolecule representation of the first biomolecule, wherein the machine-learning model comprises one or more neural networks, wherein the machine-learning model was trained using sequencing time-series data associated with biomolecule frequencies of particular biomolecules, wherein the sequencing time-series data was obtained from a directed evolution of a population of biomolecules over a plurality of enrichment rounds, wherein the plurality of enrichment rounds comprises at least three enrichment rounds, wherein at least one of the enrichment rounds was a control round where the population of biomolecules is analyzed without a presence of a target protein, wherein the population of biomolecules are amplified by polymerase chain reaction (PCR) in each of the plurality of enrichment rounds, wherein the population of biomolecules in each enrichment round was a unique set of biomolecules with respect to each other enrichment round, wherein the sequencing time-series data for each enrichment round comprises a biomolecule frequency of each biomolecule of the population of biomolecules in the respective enrichment round, wherein the sequencing time-series data comprise DNA sequencing time-series data, wherein the biomolecule frequencies of particular biomolecules indicate genotype frequencies, wherein the training comprises learning inferred fitness scores of the population of biomolecules for each enrichment round by predicting biomolecule frequencies of the population of biomolecules in the respective enrichment round given biomolecule frequencies of the population of biomolecules in one or more prior enrichment rounds, wherein learning the inferred fitness scores in the training of the machine-learning model comprises optimizing a Dirichlet-multinomial loss function, wherein the Dirichlet-multinomial loss function utilizes an over-dispersed multinomial distribution to account for an increased difficulty associated with predicting biomolecule frequencies of the population of biomolecules in each enrichment round given biomolecule frequencies of the population of biomolecules in a prior enrichment round. At step 630, the molecule discovery system 100 may output, by the machine-learning model based on the processing of the biomolecule representation of the first biomolecule, an inferred fitness score for the first biomolecule, wherein the inferred fitness score for the first biomolecule indicates a biological activity of the first biomolecule with respect to a target protein, wherein the inferred fitness score for the first biomolecule comprises one or more of an on-target fitness score associated the first biomolecule binding to a target protein or an off-target fitness score associated the first biomolecule binding to a test instrument instead of the target protein. At step 640, the molecule discovery system 100 may determine, based on the inferred fitness score for the first biomolecule, whether a biological activity associated with the first biomolecule meets a predetermined criteria for selection. Particular embodiments may repeat one or more steps of the method of FIG. 6, where appropriate. Although this disclosure describes and illustrates particular steps of the method of FIG. 6 as occurring in a particular order, this disclosure contemplates any suitable steps of the method of FIG. 6 occurring in any suitable order. Moreover, although this disclosure describes and illustrates an example method for biomolecule fitness inference, including the particular steps of the method of FIG. 6, this disclosure contemplates any suitable method for biomolecule fitness inference, including any suitable steps, which may include all, some, or none of the steps of the method of FIG. 6, where appropriate. Furthermore, although this disclosure describes and illustrates particular components, devices, or systems carrying out particular steps of the method of FIG. 6, this disclosure contemplates any suitable combination of any suitable components, devices, or systems carrying out any suitable steps of the method of FIG. 6.

### Systems and Methods

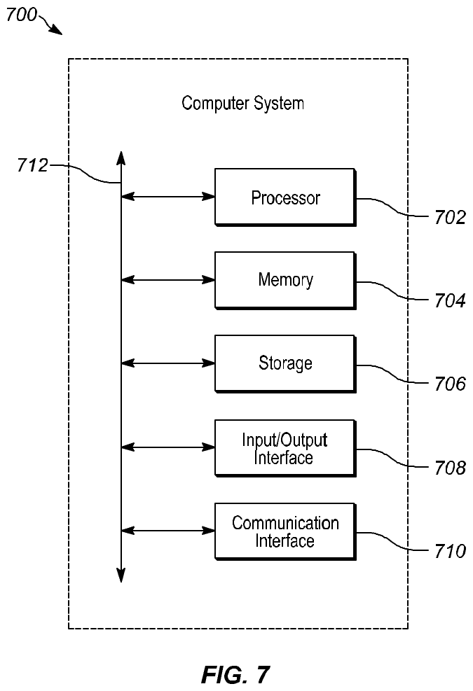

FIG. 7 illustrates an example computer system 700. In particular embodiments, one or more computer systems 700 perform one or more steps of one or more methods described or illustrated herein. In particular embodiments, one or more computer systems 700 provide functionality described or illustrated herein. In particular embodiments, software running on one or more computer systems 700 performs one or more steps of one or more methods described or illustrated herein or provides functionality described or illustrated herein. Particular embodiments include one or more portions of one or more computer systems 700. Herein, reference to a computer system may encompass a computing device, and vice versa, where appropriate. Moreover, reference to a computer system may encompass one or more computer systems, where appropriate.

This disclosure contemplates any suitable number of computer systems 700. This disclosure contemplates computer system 700 taking any suitable physical form. As example and not by way of limitation, computer system 700 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 700 may include one or more computer systems 700; be unitary or distributed; span multiple locations; span multiple machines; span multiple data centers; or reside in a cloud, which may include one or more cloud components in one or more networks. Where appropriate, one or more computer systems 700 may perform without substantial spatial or temporal limitation one or more steps of one or more methods described or illustrated herein. As an example and not by way of limitation, one or more computer systems 700 may perform in real time or in batch mode one or more steps of one or more methods described or illustrated herein. One or more computer systems 700 may perform at different times or at different locations one or more steps of one or more methods described or illustrated herein, where appropriate.

In particular embodiments, computer system 700 includes a processor 702, memory 704, storage 706, an input/output (I/O) interface 708, a communication interface 710, and a bus 712. Although this disclosure describes and illustrates a particular computer system having a particular number of particular components in a particular arrangement, this disclosure contemplates any suitable computer system having any suitable number of any suitable components in any suitable arrangement.

In particular embodiments, processor 702 includes hardware for executing instructions, such as those making up a computer program. As an example and not by way of limitation, to execute instructions, processor 702 may retrieve (or fetch) the instructions from an internal register, an internal cache, memory 704, or storage 706; decode and execute them; and then write one or more results to an internal register, an internal cache, memory 704, or storage 706. In particular embodiments, processor 702 may include one or more internal caches for data, instructions, or addresses. This disclosure contemplates processor 702 including any suitable number of any suitable internal caches, where appropriate. As an example and not by way of limitation, processor 702 may include one or more instruction caches, one or more data caches, and one or more translation lookaside buffers (TLBs). Instructions in the instruction caches may be copies of instructions in memory 704 or storage 706, and the instruction caches may speed up retrieval of those instructions by processor 702. Data in the data caches may be copies of data in memory 704 or storage 706 for instructions executing at processor 702 to operate on; the results of previous instructions executed at processor 702 for access by subsequent instructions executing at processor 702 or for writing to memory 704 or storage 706; or other suitable data. The data caches may speed up read or write operations by processor 702. The TLBs may speed up virtual-address translation for processor 702. In particular embodiments, processor 702 may include one or more internal registers for data, instructions, or addresses. This disclosure contemplates processor 702 including any suitable number of any suitable internal registers, where appropriate. Where appropriate, processor 702 may include one or more arithmetic logic units (ALUs); be a multi-core processor; or include one or more processors 702. Although this disclosure describes and illustrates a particular processor, this disclosure contemplates any suitable processor.

In particular embodiments, memory 704 includes main memory for storing instructions for processor 702 to execute or data for processor 702 to operate on. As an example and not by way of limitation, computer system 700 may load instructions from storage 706 or another source (such as, for example, another computer system 700) to memory 704. Processor 702 may then load the instructions from memory 704 to an internal register or internal cache. To execute the instructions, processor 702 may retrieve the instructions from the internal register or internal cache and decode them. During or after execution of the instructions, processor 702 may write one or more results (which may be intermediate or final results) to the internal register or internal cache. Processor 702 may then write one or more of those results to memory 704. In particular embodiments, processor 702 executes only instructions in one or more internal registers or internal caches or in memory 704 (as opposed to storage 706 or elsewhere) and operates only on data in one or more internal registers or internal caches or in memory 704 (as opposed to storage 706 or elsewhere). One or more memory buses (which may each include an address bus and a data bus) may couple processor 702 to memory 704. Bus 712 may include one or more memory buses, as described below. In particular embodiments, one or more memory management units (MMUs) reside between processor 702 and memory 704 and facilitate accesses to memory 704 requested by processor 702. In particular embodiments, memory 704 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 704 may include one or more memories 704, where appropriate. Although this disclosure describes and illustrates particular memory, this disclosure contemplates any suitable memory.

In particular embodiments, storage 706 includes mass storage for data or instructions. As an example and not by way of limitation, storage 706 may include a hard disk drive (HDD), a floppy disk drive, flash memory, an optical disc, a magneto-optical disc, magnetic tape, or a Universal Serial Bus (USB) drive or a combination of two or more of these. Storage 706 may include removable or non-removable (or fixed) media, where appropriate. Storage 706 may be internal or external to computer system 700, where appropriate. In particular embodiments, storage 706 is non-volatile, solid-state memory. In particular embodiments, storage 706 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 706 taking any suitable physical form. Storage 706 may include one or more storage control units facilitating communication between processor 702 and storage 706, where appropriate. Where appropriate, storage 706 may include one or more storages 706. Although this disclosure describes and illustrates particular storage, this disclosure contemplates any suitable storage.

In particular embodiments, I/O interface 708 includes hardware, software, or both, providing one or more interfaces for communication between computer system 700 and one or more I/O devices. Computer system 700 may include one or more of these I/O devices, where appropriate. One or more of these I/O devices may enable communication between a person and computer system 700. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 708 for them. Where appropriate, I/O interface 708 may include one or more device or software drivers enabling processor 702 to drive one or more of these I/O devices. I/O interface 708 may include one or more I/O interfaces 708, where appropriate. Although this disclosure describes and illustrates a particular I/O interface, this disclosure contemplates any suitable I/O interface.

In particular embodiments, communication interface 710 includes hardware, software, or both providing one or more interfaces for communication (such as, for example, packet-based communication) between computer system 700 and one or more other computer systems 700 or one or more networks. As an example and not by way of limitation, communication interface 710 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 710 for it. As an example and not by way of limitation, computer system 700 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 700 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 700 may include any suitable communication interface 710 for any of these networks, where appropriate. Communication interface 710 may include one or more communication interfaces 710, where appropriate. Although this disclosure describes and illustrates a particular communication interface, this disclosure contemplates any suitable communication interface.

In particular embodiments, bus 712 includes hardware, software, or both coupling components of computer system 700 to each other. As an example and not by way of limitation, bus 712 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 712 may include one or more buses 712, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (Ics) (such, as for example, field-programmable gate arrays (FPGAs) or application-specific Ics (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

### Recitation of Embodiments

Embodiment 1: A method including, by one or more computing systems: accessing a biomolecule representation of a first biomolecule; processing, by a machine-learning model, the biomolecule representation of the first biomolecule, wherein the machine-learning model was trained using sequencing time-series data associated with biomolecule frequencies of particular biomolecules, wherein the sequencing time-series data was obtained from directed evolution of a population of biomolecules over a plurality of enrichment rounds, wherein the population of biomolecules in each enrichment round was a unique set of biomolecules with respect to each other enrichment round, wherein the sequencing time-series data for each enrichment round includes a biomolecule frequency of each biomolecule of the population of biomolecules in the respective enrichment round, and wherein the training includes learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and outputting, by the machine-learning model based on the processing of the biomolecule representation of the first biomolecule, an inferred fitness score for the first biomolecule.

Embodiment 2: The method of Embodiment 1, further including: determining, based on the inferred fitness score for the first biomolecule, whether a biological activity associated with the first biomolecule meets a predetermined criteria for selection.

Embodiment 3: The method of either of Embodiments 1-2, wherein the plurality of enrichment rounds includes at least three enrichment rounds, and wherein at least one of the enrichment rounds was a control round where the population of biomolecules is analyzed without a presence of a target protein.

Embodiment 4: The method of any one of Embodiments 1-3, wherein the inferred fitness score for the first biomolecule indicates a biological activity of the first biomolecule with respect to a target protein.

Embodiment 5: The method of any one of Embodiments 1-4, wherein learning the inferred fitness scores in the training of the machine-learning model includes optimizing a Dirichlet-multinomial loss function, and wherein the Dirichlet-multinomial loss function utilizes an over-dispersed multinomial distribution to account for an increased difficulty associated with predicting biomolecule frequencies of the population of biomolecules in each enrichment round given biomolecule frequencies of the population of biomolecules in a prior enrichment round.

Embodiment 6: The method of any one of Embodiments 1-5, wherein the training of the machine-learning model further includes: calculating a Dirichlet loss negative log-likelihood between the predicted biomolecule frequencies and actual biomolecule frequencies as a negative log-likelihood.

Embodiment 7: The method of any one of Embodiments 1-6, wherein the inferred fitness score for the first biomolecule includes an on-target fitness score associated the first biomolecule binding to a target protein.

Embodiment 8: The method of any one of Embodiments 1-6, wherein the inferred fitness score for the first biomolecule includes an off-target fitness score associated the first biomolecule binding to a test instrument instead of a target protein.

Embodiment 9: The method of any one of Embodiments 1-6, wherein the inferred fitness score for the first biomolecule includes an on-target fitness score associated the first biomolecule binding to a target protein and an off-target fitness score associated the first biomolecule binding to a test instrument instead of the target protein, wherein the method further includes: determining a binding specificity of the first biomolecule based on a ratio of the on-target fitness score to the off-target fitness score.

Embodiment 10: The method of any one of Embodiments 1-9, wherein the machine-learning model includes one or more neural networks.

Embodiments 11: The method of Embodiment 10, wherein the one or more neural networks include: a first neural network trained for predicting on-target fitness scores associated with biomolecules, and a second neural network trained for predicting off-target fitness scores associated with biomolecules.

Embodiment 12: The method of any one of Embodiments 1-11, further including: generating the biomolecule representation of the first biomolecule, wherein the first biomolecule is a polypeptide corresponding to a first genotype, and wherein the generating includes: determining a plurality of amino acids of the first biomolecule; applying, for each amino acid of the plurality of amino acids, a function to determine a feature representation for the respective amino acid; and generating a genotype representation corresponding to the first genotype based on the plurality of feature representations associated with the plurality of amino acids.

Embodiment 13: The method of any one of Embodiments 1-12, wherein the first biomolecule is within the population of biomolecules in the plurality of enrichment rounds.

Embodiment 14: The method of any one of Embodiments 1-12, wherein the first biomolecule is not within the population of biomolecules in the plurality of enrichment rounds.

Embodiment 15: The method of any one of Embodiments 1-14, wherein the sequencing time-series data include DNA sequencing time-series data, and wherein the biomolecule frequencies of particular biomolecules indicate genotype frequencies.

Embodiment 16: The method of any one of Embodiments 1-15, further including: processing a plurality of biomolecule representations associated with a plurality of respective second biomolecules by the machine-learning model to determine a plurality of inferred fitness scores for the plurality of second biomolecules, respectively; and selecting, based on the inferred fitness scores for the plurality of second biomolecules, one or more second biomolecules meeting a predetermined criteria for selection, wherein one or more of the selected second biomolecules are each associated with a low relative biomolecule frequency in a last round of the plurality of enrichment rounds.

Embodiment 17: The method of Embodiment 16, further including: generating a genotype space based on the biomolecule frequencies and the inferred fitness scores for the plurality of second biomolecules; and selecting the one or more second biomolecules by identifying the one or more second biomolecules from one or more regions in the genotype space, wherein each of the one or more regions is associated with a particular biomolecule frequency range and a particular biomolecule fitness range.

Embodiment 18: The method of any one of Embodiments 1-17, wherein the training further includes pretraining an off-target model, including: identifying one or more off-target enrichment rounds from the plurality of enrichment rounds; and pretraining the off-target model based on sequencing time-series data for the one or more off-target enrichment rounds.

Embodiment 19: The method of Embodiment 18, wherein the training further includes: accessing sequencing time-series data from one or more on-target enrichment rounds from the plurality of enrichment rounds; and generating an on-target model based on the accessed sequencing time-series data from the one or more on-target enrichment rounds and the off-target model.

Embodiment 20: The method of any one of Embodiments 1-19, wherein the first biomolecule is a macrocycle.

Embodiment 21: The method of any one of Embodiments 1-20, wherein the population of biomolecules are amplified by polymerase chain reaction (PCR) in each of the plurality of enrichment rounds.

Embodiment 22: The method of any one of Embodiments 1-21, further including: processing a plurality of biomolecule representations associated with a plurality of respective second biomolecules by the machine-learning model to determine a plurality of inferred fitness scores for the plurality of second biomolecules, respectively; and selecting, based on the inferred fitness scores for the plurality of second biomolecules, one or more diverse biomolecules from the plurality of second biomolecules, wherein the one or more diverse biomolecules meet a predetermined criteria for selection, and wherein one or more of the diverse second biomolecules are each associated with a low relative biomolecule frequency in a last round of the plurality of enrichment rounds

Embodiment 23: One or more computer-readable non-transitory storage media embodying software that is operable when executed to: access a biomolecule representation of a first biomolecule; process, by a machine-learning model, the biomolecule representation of the first biomolecule, wherein the machine-learning model was trained using sequencing time-series data associated with biomolecule frequencies of particular biomolecules, wherein the sequencing time-series data was obtained from a directed evolution of a population of biomolecules over a plurality of enrichment rounds, wherein the population of biomolecules in each enrichment round was a unique set of biomolecules with respect to each other enrichment round, wherein the sequencing time-series data for each enrichment round includes a biomolecule frequency of each biomolecule of the population of biomolecules in the respective enrichment round, and wherein the training includes learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and output, by the machine-learning model based on the processing of the biomolecule representation of the first biomolecule, an inferred fitness score for the first biomolecule.

Embodiment 24: A system including: one or more processors; and a non-transitory memory coupled to the processors including instructions executable by the processors, the processors operable when executing the instructions to: access a biomolecule representation of a first biomolecule; process, by a machine-learning model, the biomolecule representation of the first biomolecule, wherein the machine-learning model was trained using sequencing time-series data associated with biomolecule frequencies of particular biomolecules, wherein the sequencing time-series data was obtained from a directed evolution of a population of biomolecules over a plurality of enrichment rounds, wherein the population of biomolecules in each enrichment round was a unique set of biomolecules with respect to each other enrichment round, wherein the sequencing time-series data for each enrichment round includes a biomolecule frequency of each biomolecule of the population of biomolecules in the respective enrichment round, and wherein the training includes learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and output, by the machine-learning model based on the processing of the biomolecule representation of the first biomolecule, an inferred fitness score for the first biomolecule.

Embodiment 25: A method including, by one or more computing systems: accessing a plurality of biomolecule representations of a plurality of respective biomolecules; processing, by a machine-learning model, the plurality of biomolecule representations of the plurality of respective biomolecules, wherein the machine-learning model was trained using sequencing time-series data associated with biomolecule frequencies of particular biomolecules, wherein the sequencing time-series data was obtained from directed evolution of a population of biomolecules over a plurality of enrichment rounds, wherein the population of biomolecules in each enrichment round was a unique set of biomolecules with respect to each other enrichment round, wherein the sequencing time-series data for each enrichment round includes a biomolecule frequency of each biomolecule of the population of biomolecules in the respective enrichment round, and wherein the training includes learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; outputting, by the machine-learning model based on the processing of the plurality of biomolecule representation of the plurality of biomolecules, a plurality of inferred fitness scores for the plurality of biomolecules, respectively; and selecting, based on the inferred fitness scores for the plurality of biomolecules, one or more biomolecules meeting a predetermined criteria for selection, wherein one or more of the selected biomolecules are each associated with a low relative biomolecule frequency in a last round of the plurality of enrichment rounds.

Embodiment 26: The method of Embodiment 25, further including: generating a genotype space based on the biomolecule frequencies and the inferred fitness scores for the plurality of biomolecules, wherein selecting the one or more biomolecules meeting the predetermined criteria for selection comprises identifying the one or more biomolecules from one or more regions in the genotype space, wherein each of the one or more regions is associated with a particular biomolecule frequency range and a particular biomolecule fitness range . . .

Embodiment 27: The method of either of Embodiments 25-26, further including: determining, based on an inferred fitness score for a first biomolecule of the plurality of biomolecules, whether a biological activity associated with the first biomolecule meets a predetermined criteria for selection.

Embodiment 28: The method of any one of Embodiments 25-27, wherein the plurality of enrichment rounds includes at least three enrichment rounds, and wherein at least one of the enrichment rounds was a control round where the population of biomolecules is analyzed without a presence of a target protein.

Embodiment 29: The method of any one of Embodiments 25-28, wherein the inferred fitness score for each of the plurality of biomolecules indicates a biological activity of the corresponding biomolecule with respect to a target protein.

Embodiment 30: The method of any one of Embodiments 25-29, wherein learning the inferred fitness scores in the training of the machine-learning model includes optimizing a Dirichlet-multinomial loss function, and wherein the Dirichlet-multinomial loss function utilizes an over-dispersed multinomial distribution to account for an increased difficulty associated with predicting biomolecule frequencies of the population of biomolecules in each enrichment round given biomolecule frequencies of the population of biomolecules in a prior enrichment round.

Embodiment 31: The method of any one of Embodiments 25-30, wherein the training of the machine-learning model further includes: calculating a Dirichlet loss negative log-likelihood between the predicted biomolecule frequencies and actual biomolecule frequencies as a negative log-likelihood.

Embodiment 32: The method of any one of Embodiments 25-31, wherein the inferred fitness score for each of the plurality of biomolecules includes an on-target fitness score associated the corresponding biomolecule binding to a target protein.

Embodiment 33: The method of any one of Embodiments 25-31, wherein the inferred fitness score for each of the plurality of biomolecules includes an off-target fitness score associated the corresponding biomolecule binding to a test instrument instead of a target protein.

Embodiment 34: The method of any one of Embodiments 25-31, wherein the inferred fitness score for each of the plurality of biomolecules includes an on-target fitness score associated the corresponding biomolecule binding to a target protein and an off-target fitness score associated the corresponding biomolecule binding to a test instrument instead of the target protein, wherein the method further includes: determining a binding specificity of the corresponding biomolecule based on a ratio of the on-target fitness score to the off-target fitness score.

Embodiment 35: The method of any one of Embodiments 25-34, wherein the machine-learning model includes one or more neural networks.

Embodiments 36: The method of Embodiment 35, wherein the one or more neural networks include: a first neural network trained for predicting on-target fitness scores associated with biomolecules, and a second neural network trained for predicting off-target fitness scores associated with biomolecules.

Embodiment 37: The method of any one of Embodiments 25-36, further including: generating the biomolecule representation of a first biomolecule of the plurality of biomolecules, wherein the first biomolecule is a polypeptide corresponding to a first genotype, and wherein the generating includes: determining a plurality of amino acids of the first biomolecule; applying, for each amino acid of the plurality of amino acids, a function to determine a feature representation for the respective amino acid; and generating a genotype representation corresponding to the first genotype based on the plurality of feature representations associated with the plurality of amino acids.

Embodiment 38: The method of any one of Embodiments 25-37, wherein each of the plurality of biomolecules is within the population of biomolecules in the plurality of enrichment rounds.

Embodiment 39: The method of any one of Embodiments 25-37, wherein each of the plurality of biomolecules is not within the population of biomolecules in the plurality of enrichment rounds.

Embodiment 40: The method of any one of Embodiments 25-39, wherein the sequencing time-series data include DNA sequencing time-series data, and wherein the biomolecule frequencies of particular biomolecules indicate genotype frequencies.

Embodiment 41: The method of any one of Embodiments 25-40, wherein the training further includes pretraining an off-target model, including: identifying one or more off-target enrichment rounds from the plurality of enrichment rounds; and pretraining the off-target model based on sequencing time-series data for the one or more off-target enrichment rounds.

Embodiment 42: The method of Embodiment 41, wherein the training further includes: accessing sequencing time-series data from one or more on-target enrichment rounds from the plurality of enrichment rounds; and generating an on-target model based on the accessed sequencing time-series data from the one or more on-target enrichment rounds and the off-target model.

Embodiment 43: The method of any one of Embodiments 25-42, wherein a first biomolecule of the plurality of biomolecules is a macrocycle.

Embodiment 44: The method of any one of Embodiments 25-43, wherein the population of biomolecules are amplified by polymerase chain reaction (PCR) in each of the plurality of enrichment rounds.

### Miscellaneous

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A method comprising, by one or more computing systems:
accessing a biomolecule representation of a first biomolecule;
processing, by a machine-learning model, the biomolecule representation of the first biomolecule,
wherein the machine-learning model was trained using sequencing time-series data associated with biomolecule frequencies of particular biomolecules,
wherein the sequencing time-series data was obtained from directed evolution of a population of biomolecules over a plurality of enrichment rounds,
wherein the population of biomolecules in each enrichment round was a unique set of biomolecules with respect to each other enrichment round,
wherein the sequencing time-series data for each enrichment round comprises a biomolecule frequency of each biomolecule of the population of biomolecules in the respective enrichment round, and
wherein the training comprises learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and
outputting, by the machine-learning model based on the processing of the biomolecule representation of the first biomolecule, an inferred fitness score for the first biomolecule.

2. The method of claim 1, further comprising:
determining, based on the inferred fitness score for the first biomolecule, whether a biological activity associated with the first biomolecule meets a predetermined criteria for selection.

3. The method of claim 1, wherein the plurality of enrichment rounds comprises at least three enrichment rounds, and wherein at least one of the enrichment rounds was a control round where the population of biomolecules is analyzed without a presence of a target protein.

4. The method of claim 1, wherein the inferred fitness score for the first biomolecule indicates a biological activity of the first biomolecule with respect to a target protein.

5. The method of claim 1, wherein learning the inferred fitness scores in the training of the machine-learning model comprises optimizing a Dirichlet-multinomial loss function, and wherein the Dirichlet-multinomial loss function utilizes an over-dispersed multinomial distribution to account for an increased difficulty associated with predicting biomolecule frequencies of the population of biomolecules in each enrichment round given biomolecule frequencies of the population of biomolecules in a prior enrichment round.

6. The method of claim 5, wherein the training of the machine-learning model further comprises:
calculating a Dirichlet loss negative log-likelihood between the predicted biomolecule frequencies and actual biomolecule frequencies as a negative log-likelihood.

7. The method of claim 1, wherein the inferred fitness score for the first biomolecule comprises one or more of an on-target fitness score associated the first biomolecule binding to a target protein or an off-target fitness score associated the first biomolecule binding to a test instrument instead of the target protein.

8. The method of claim 1, wherein the inferred fitness score for the first biomolecule comprises an on-target fitness score associated the first biomolecule binding to a target protein and an off-target fitness score associated the first biomolecule binding to a test instrument instead of the target protein, wherein the method further comprises:
determining a binding specificity of the first biomolecule based on a ratio of the on-target fitness score to the off-target fitness score.

9. The method of claim 1, wherein the machine-learning model comprising one or more neural networks comprising:
a first neural network trained for predicting on-target fitness scores associated with biomolecules, and
a second neural network trained for predicting off-target fitness scores associated with biomolecules.

10. The method of claim 1, further comprising:
generating the biomolecule representation of the first biomolecule, wherein the first biomolecule is a polypeptide corresponding to a first genotype, and wherein the generating comprises:
determining a plurality of amino acids of the first biomolecule;
applying, for each amino acid of the plurality of amino acids, a function to determine a feature representation for the respective amino acid; and
generating a genotype representation corresponding to the first genotype based on the plurality of feature representations associated with the plurality of amino acids.

11. The method of claim 1, wherein the sequencing time-series data comprise DNA sequencing time-series data, and wherein the biomolecule frequencies of particular biomolecules indicate genotype frequencies.

12. The method of claim 1, further comprising:
processing a plurality of biomolecule representations associated with a plurality of respective second biomolecules by the machine-learning model to determine a plurality of inferred fitness scores for the plurality of second biomolecules, respectively; and
selecting, based on the inferred fitness scores for the plurality of second biomolecules, one or more second biomolecules meeting a predetermined criteria for selection, wherein one or more of the selected second biomolecules are each associated with a low relative biomolecule frequency in a last round of the plurality of enrichment rounds.

13. The method of claim 12, further comprising:
generating a genotype space based on the biomolecule frequencies and the inferred fitness scores for the plurality of second biomolecules; and
selecting the one or more second biomolecules by identifying the one or more second biomolecules from one or more regions in the genotype space, wherein each of the one or more regions is associated with a particular biomolecule frequency range and a particular biomolecule fitness range.

14. The method of claim 1, wherein the training further comprises pretraining an off-target model, comprising:
identifying one or more off-target enrichment rounds from the plurality of enrichment rounds; and
pretraining the off-target model based on sequencing time-series data for the one or more off-target enrichment rounds.

15. The method of claim 14, wherein the training further comprises:
accessing sequencing time-series data from one or more on-target enrichment rounds from the plurality of enrichment rounds; and
generating an on-target model based on the accessed sequencing time-series data from the one or more on-target enrichment rounds and the off-target model.

16. The method of claim 1, wherein the first biomolecule is a macrocycle.

17. The method of claim 1, wherein the population of biomolecules are amplified by polymerase chain reaction (PCR) in each of the plurality of enrichment rounds.

18. The method of claim 1, further comprising:
processing a plurality of biomolecule representations associated with a plurality of respective second biomolecules by the machine-learning model to determine a plurality of inferred fitness scores for the plurality of second biomolecules, respectively; and
selecting, based on the inferred fitness scores for the plurality of second biomolecules, one or more diverse biomolecules from the plurality of second biomolecules, wherein the one or more diverse biomolecules meet a predetermined criteria for selection, and wherein one or more of the diverse biomolecules are each associated with a low relative biomolecule frequency in a last round of the plurality of enrichment rounds.

19. One or more computer-readable non-transitory storage media embodying software that is operable when executed to:
access a biomolecule representation of a first biomolecule;
process, by a machine-learning model, the biomolecule representation of the first biomolecule,
wherein the machine-learning model was trained using sequencing time-series data associated with biomolecule frequencies of particular biomolecules,
wherein the sequencing time-series data was obtained from a directed evolution of a population of biomolecules over a plurality of enrichment rounds,
wherein the population of biomolecules in each enrichment round was a unique set of biomolecules with respect to each other enrichment round,
wherein the sequencing time-series data for each enrichment round comprises a biomolecule frequency of each biomolecule of the population of biomolecules in the respective enrichment round, and
wherein the training comprises learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and
output, by the machine-learning model based on the processing of the biomolecule representation of the first biomolecule, an inferred fitness score for the first biomolecule.

20. A system comprising: one or more processors; and a non-transitory memory coupled to the processors comprising instructions executable by the processors, the processors operable when executing the instructions to:
access a biomolecule representation of a first biomolecule;
process, by a machine-learning model, the biomolecule representation of the first biomolecule,
wherein the machine-learning model was trained using sequencing time-series data associated with biomolecule frequencies of particular biomolecules,
wherein the sequencing time-series data was obtained from a directed evolution of a population of biomolecules over a plurality of enrichment rounds,
wherein the population of biomolecules in each enrichment round was a unique set of biomolecules with respect to each other enrichment round,
wherein the sequencing time-series data for each enrichment round comprises a biomolecule frequency of each biomolecule of the population of biomolecules in the respective enrichment round, and
wherein the training comprises learning inferred fitness scores of the population of biomolecules for each enrichment round by predicting biomolecule frequencies of the population of biomolecules in the respective enrichment round given biomolecule frequencies of the population of biomolecules in one or more prior enrichment rounds; and
output, by the machine-learning model based on the processing of the biomolecule representation of the first biomolecule, an inferred fitness score for the first biomolecule.
